(19) United States
(12) Patent Application Publication
(10) Pub. No.: US 2006/0110364 A1
(43) Pub. Date: May 25, 2006

(54) Vector-mediated delivery of polynucleotides encoding soluble VEGF receptors

(75) Inventor: Thomas Harding, San Francisco, CA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: LUDWIG INSTITUTE FOR CANCER RESEARCH, New York, NY (US)

(21) Appl. No.: 11/269,117

(22) Filed: Nov. 8, 2005

Related U.S. Application Data
(63) Continuation-in-part of application No. 11/208,696, filed on Aug. 22, 2005.
(60) Provisional application No. 60/602,926, filed on Aug. 20, 2004.

(51) Int. Cl. A61K 48/00; C12N 15/86

(52) U.S. Cl. 424/93.2; 435/456

(57) ABSTRACT

The present invention provides vector compositions for expression of a soluble form of VEGFR3 and methods for their use in the inhibition of one or more of lymphangiogenesis, lymphatic metastasis and angiogenesis, as a therapeutic strategy for treatment of cancer.

The present application is a continuation-in-part of U.S. patent application Ser. No. 11/208,696, which was filed on Aug. 22, 2005, which claims the benefit of priority to U.S. Provisional Application No. 60/602,926, which was filed on Aug. 20, 2004. The entire text of the priority applications are incorporated herein by reference in their entirety.

### BACKGROUND OF THE INVENTION

1. Field of the Invention

The present invention relates to vector-mediated delivery and expression of polynucleotides encoding soluble VEGF receptors. More specifically, the invention relates to the use of recombinant viral vectors to deliver genes encoding sVEGFR3 for use in the inhibition of lymphangiogenesis, lymphatic metastasis and angiogenesis as a therapeutic strategy for treatment of cancer.

2. Background of the Technology

It is generally accepted that tumor development requires the secretion of soluble mediators by cancer cells, so-called “tumor angiogenic factors”, which activate the formation of new blood vessels. The discovery of vascular endothelial growth factor (VEGF) and of new proteases had led to the identification of the key factors involved in tumor angiogenesis. The elucidation of their mechanisms of action has allowed the design of new therapeutic strategies, including the use of anti-angiogenic compounds.

Metastatic spread of cancer cells through the bloodstream or lymphatic vessels is a hallmark of malignancy. The presence of metastases in regional lymph nodes is an important clinical indicator for tumor progression in many types of cancer. However, very little is known about the mechanism of development of lymph node metastasis. Recent studies have demonstrated that a member of the vascular endothelial growth factor, VEGF-C (or VEGFC, both abbreviations are used interchangeably herein), can bind to its receptor, VEGFR3 (or VEGFR-3, both abbreviations are used interchangeably herein), which is predominantly expressed in endothelium of lymphatic vessels and stimulates lymphangiogenesis and lymphatic endothelial cell growth, migration, and survival (Karpanen t et al., Cancer Res. 2001 Mar. 1;61(5):1786-90; Marika J and Alitalo K, Cell & Dev. Biol. 2002). VEGF-C has been shown to be a critical mediator of lymphangiogenesis during development and in inducing tumor-associated lymphangiogenesis that promotes lymphatic-mediated metastasis (Stacker, S. A. et al, FASEB J 2002, Alitalo et al, Nat. Med 2002, Madriota S J, et al, EMBO 2001, Karkkainen M J & Petrova T V Oncogene 2000) in several types of solid cancers including gastric (Liu, X et al, World J Gastro. 2004, Yonemura, Y et al, Cli. Cancer Res. 1999), prostate cancer (Tsurusaki, T et al, Br. J. Cancer, 1999), colon (Akagi, K, Br. J. Cancer, 2000), cervival cancer (Hashimoto, I, Br. J. Cancer, 2001), breast cancer (Skobe M, et al, Nat. Med 2001), thyroid cancer (Shushanov S, et al, Int J Cancer. 2000) and melanoma (Schietroma, C, Cancer, 2003). The degree of tumor lymphangiogenesis has been observed to correlate with the extent of lymph node and lung metastases (Skobe M, et al, Nat. Med. 2001). In addition, suppression of VEGF-C with neutralizing antibodies or blockade of the VEGFR3 signaling pathways by a soluble VEGFR3-Ig fusion protein in tumor-bearing mice has been shown to suppress tumor lymphangiogenesis thereby resulting in the inhibition of tumor metastasis to regional lymph nodes (Karpanen et al, J. Exp. Med, 2001, Makinen et al, Nat. Med, 2001, Stacker et al, Nat. Med, 2001, He Y et al, J. Nat. Cancer Ins. 2002; International Patent Publication No. WO 00/21560; and International Patent Publication No. WO 02/060950). Numerous cancer treatment strategies that rely on inhibition of a component of the anigogenesis process are in various stages of clinical development.

Vector-mediated delivery of therapeutic agents provides the advantage of a relatively constant level of the agent in vivo and is therefore able to improve the therapeutic efficacy of a given agent in tumor models, as compared to cyclical delivery of the same agent.

Despite advances in cancer treatment strategies, lack of efficacy and/or significant side effects due to the toxicity of currently used chemotherapeutic agents remains a problem. Toxicity associated with chemotherapy can be severe enough to result in life-threatening situations, which require administration of drugs to counteract the side effects, and may result in the reduction and/or discontinuation of chemotherapy, which can impact negatively on the patient's treatment and/or quality of life. Gene therapy strategies have been attempted and are the subject of ongoing clinical trials, but have not yet proven to have clinical usefulness. Accordingly, there remains a need for improved cancer treatment regimens which address the deficiencies in current therapeutic approaches. The present invention addresses this need. More specifically, there is currently a need for vectors and methods that allow for delivery and expression of compounds effective against lymphangiogenesis, lymphatic metastasis and angiogenesis in general such that persistent in vivo expression is accomplished with minimal side effects to the subject under treatment.

### SUMMARY OF THE INVENTION

The invention provides composition and methods for vector-mediated delivery of soluble vascular endothelial growth factor 3 (sVEGFR3), which consists of the ligand-binding domains of VEGFR3 receptor fused to a human IgG Fc sequence.

Typically the vector comprises the coding sequence for a soluble form of VEGFR3 operably linked to a promoter. In one aspect the vector is a viral vector, such as an adeno-associated virus (AAV) vector.

In a further aspect, the promoter is the cytomegalovirus enhancer/chicken beta-actin/Rabbit β-globin promoter (CAG) promoter.

The invention also provides methods for expressing sVEGFR3 and methods for inhibiting lymphangiogenesis, lymphatic metastasis and angiogenesis in a mammalian subject, by administering a viral vector comprising a DNA sequence encoding a soluble form of VEGFR3 operably linked to a promoter, in a manner effective to result in expression of a biologically active soluble form of VEGFR3.

In one aspect of the invention, the vector is administered in vivo: intramuscularly, intravenously or into the portal vasculature of the mammal.

Additional features and variations of the invention will be apparent to those skilled in the art from the entirety of this application, including the detailed description, and all such features are intended as aspects of the invention. It should be understood, however, that the detailed description and the specific examples, while indicating preferred embodiments of the invention, are given by way of illustration only, because various changes and modifications within the spirit and scope of the invention will become apparent to those skilled in the art from this detailed description.

Moreover, features of the invention described herein can be re-combined into additional embodiments that also are intended as aspects of the invention, irrespective of whether the combination of features is specifically mentioned above as an aspect or embodiment of the invention. Also, only those limitations that are described herein as critical to the invention should be viewed as such; variations of the invention lacking features that have not been described herein as critical are intended as aspects of the invention.

With respect to aspects of the invention that have been described as a set or genus, every individual member of the set or genus is intended, individually, as an aspect of the invention, even if, for brevity, every individual member has not been specifically mentioned herein. When aspects of the invention that are described herein as being selected from a genus, it should be understood that the selection can include mixtures of two or more members of the genus.

In addition to the foregoing, the invention includes, as an additional aspect, all embodiments of the invention narrower in scope in any way than the variations specifically described herein. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

These and other objects and features of the invention will be more fully appreciated when the following detailed description of the invention is read in conjunction with the accompanying drawings.

### BRIEF DESCRIPTION OF THE FIGURES

FIG. 1 is a schematic depiction of soluble VEGF-receptor 3 (sVEGFR3) showing the extracellular ligand binding region of VEGFR3 fused to Fc moiety.

FIG. 2 is a schematic depiction of an exemplary AAV vector which encodes sVEGFR3 (rAAV-VEGFR-3Fc), encompassing the 1-3 Ig-like domains of human VEGFR-3 gene fused to the human IgG1 heavy chain (Fc) cloned downstream of the constitutive CAG promoter and upstream of the Woodchuck Hepatitis Virus Post-transcriptional regulatory element (WPRE) and bovine growth hormone polyadenylation sequence (bGHpA). ITRs represent the AAV-2 inverted terminal repeats.

FIG. 3 is an image of a Western blot illustrating the results of analysis of HuH7 cells transduced with soluble human AAV/VEGFR3-Fc. Conditioned media collected from AAV-Null (lane 1) and AAV-sVEGFR3-Fc transduced HuH7 cells (lane 2), or 100 ng of recombinant human VEGFR3-Fc protein (lane 3), were resolved by SDS-PAGE and probed with an anti-VEGFR3 anti-sera. Molecular weight markers are shown on the left of the panel and the arrow indicates the detection of the expressed sVEGFR3-Fc at 115 kD.

FIG. 4 illustrates the results of dose-dependent inhibition of VEGFC-mediated cell proliferation by a soluble form of human VEGFR3-Fc expressed by way of an AAV vector.

FIG. 5 illustrates long-term sustained expression of soluble human VEGFR3-Fc in vivo following AAV-mediated gene transfer in immune-deficient mice.

FIG. 6 illustrates the effect of systemic expression of AAV-sVEGFR3-Fc on PC-3-mlg2 tumor metastasis to the lymph nodes in immune-deficient mice. Each data point represents the total bioluminescence (CCD counts) of all six lymph nodes (including axillaries and inguinal from both sides) collected from each animal.

FIG. 7 illustrates the dose-dependent effect of AAV-sVEGFR3-Fc expression on inhibition of A375 tumor metastasis to the lymph nodes in immune-deficient mice. Each data point represents the total bioluminescence (CCD counts) of all six lymph nodes (including axillaries and inguinal from both sides) collected from each animal.

FIG. 8 illustrates the results of a study showing that AAV-sVEGFR3-Fc serum levels that block tumor-induced lymph node metastasis are dependent upon the amount of VEGF-C expressed by the primary tumor. Each data point represents the total bioluminescence (CCD counts) of all six lymph nodes (including axillaries and inguinal from both sides) collected from each animal.

FIG. 9 illustrates the effect of AAV-sVEGFR3-Fc on Caki-2 tumor metastasis to the lymph nodes in immune deficient mice. Each data point represents the total volume of all six lymph nodes (including axillaries and inguinal from both sides) collected from each animal.

### DETAILED DESCRIPTION OF THE INVENTION

The present invention addresses the effects of vector-mediated expression of a soluble VEGF-C/D inhibitor on tumor lymphangiogenesis, lymphatic metastasis and angiogenesis in vitro and in vivo.

Definitions

Unless otherwise indicated, all technical and scientific terms used herein have the same meaning as they would to one skilled in the art of the present invention. Practitioners are particularly directed to Sambrook et al., Molecular Cloning: A Laboratory Manual (Second Edition), Cold Spring Harbor Press, Plainview, N.Y., 1989 and Ausubel F M et al., Current Protocols in Molecular Biology, John Wiley & Sons, New York, N.Y. 1993, for definitions and terms of the art. It is to be understood that this invention is not limited to the particular methodology, protocols, and reagents described, as these may vary.

The publications and other materials including all patents, patent applications, publications (including published patent applications), and database accession numbers referred to in this specification are used herein to illuminate the background of the invention and in particular, cases to provide additional details respecting the practice. The publications and other materials including all patents, patent applications, publications (including published patent applications), and database accession numbers referred to in this specification are incorporated herein by reference to the same extent as if each were specifically and individually indicated to be incorporated by reference in its entirety.

The term “vector”, as used herein, refers to a nucleic acid construct designed for transfer between different host cells. An “expression vector” or “gene therapy vector” refers to a vector that has the ability to incorporate and express heterologous DNA fragments in a foreign cell. A cloning or expression vector may comprise additional elements, for example, the expression vector may have two replication systems, thus allowing it to be maintained in two organisms, for example in human cells for expression and in a prokaryotic host for cloning and amplification. The term vector may also be used to describe a recombinant virus, e.g., a virus modified to contain the coding sequence for a therapeutic compound or factor. As used herein, a vector may be of viral or non-viral origin.

The terms “virus,” “viral particle,” “vector particle,” “viral vector particle,” and “virion” are used interchangeably and are to be understood broadly as meaning infectious viral particles that are formed when, e.g., a viral vector of the invention is transduced into an appropriate cell or cell line. Viral particles according to the invention may be utilized for the purpose of transferring DNA into cells either in vitro or in vivo. The terms “vector,” “polynucleotide vector,” “polynucleotide vector construct,” “nucleic acid vector construct,” and “vector construct” are used interchangeably herein to mean any nucleic acid construct for gene transfer, as understood by one skilled in the art.

As used herein, the term “viral vector” is used according to its art-recognized meaning. It refers to a nucleic acid vector construct that includes at least one element of viral origin and may be packaged into a viral vector particle. The vector and/or particle may be utilized for the purpose of transferring DNA, RNA or other nucleic acids into cells either in vitro or in vivo. Numerous forms of viral vectors are known in the art.

The term “nucleic acid” refers to deoxyribonucleotides or ribonucleotides and polymers thereof (“polynucleotides”) in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid molecule/polynucleotide also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); Rossolini et al., Mol. Cell. Probes 8: 91-98 (1994)). Nucleotides are indicated by their bases by the following standard abbreviations: adenine (A), cytosine (C), thymine (T), and guanine (G). As is well known, the RNA equivalent of a DNA sequence contains uracil (U) bases where DNA contains thymine.

The term “replication defective” as used herein relative to a viral gene therapy vector of the invention means the viral vector cannot further replicate and package its genomes. For example, when the cells of a subject are infected with rAAV virions, the heterologous gene is expressed in the patient's cells, however, due to the fact that the patient's cells lack AAV rep and cap genes and the adenovirus accessory function genes necessary to replicate and package rAAV, the rAAV is replication defective and wild-type AAV cannot be formed in the patient's cells.

As used herein, “packaging system” refers to a set of viral constructs comprising genes that encode viral proteins involved in packaging a recombinant virus. Typically, the constructs of the packaging system will ultimately be incorporated into a packaging cell.

The term “operably linked” as used herein relative to a recombinant DNA construct or vector means nucleotide components of the recombinant DNA construct or vector that are directly linked to one another for operative control of a selected coding sequence. Generally, “operably linked” DNA sequences are contiguous, and, in the case of a secretory leader, contiguous and in reading frame. However, enhancers do not have to be contiguous.

As used herein, the term “gene” or “coding sequence” means the nucleic acid sequence which is transcribed (DNA) and translated (mRNA) into a polypeptide in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, “transgene” refers to a polynucleotide that can be expressed, via recombinant techniques, in a non-native environment or heterologous cell under appropriate conditions. The transgene may be derived from the same type of cell in which it is to be expressed, but introduced from an exogenous source, modified as compared to a corresponding native form and/or expressed from a non-native site, or it may be derived from a heterologous cell. “Transgene” is synonymous with “exogenous gene”, “foreign gene” and “heterologous gene”.

The term “lymphangiogenesis”, as used herein refers to the formation of lymphatic vessels from pre-existing lymphatic vessels. Such lymphatic vessels may be characterized as having a single continuous layer of lymphatic endothelial cells with poorly developed junctions and frequent large interendothelial gaps. The specific expression of cellular markers including LYVE-1, Podoplanin, Prox-1 and VEGFR3/Flt-4 distinguish lymphatic vessels from blood vessels.

The term “lymphatic metastasis”, as used herein refers to tansmission of cancerous cells from a primary growth site to one or more sites elsewhere in the body, by way of the lymphatic system. Without wishing to be bound by theory or mechanism, it is generally understood that afferent lymphatic vessels serve as a conduit of tumor cells to sentinel lymph nodes and that subsequent metastatic spread to distal sites may result from the trafficking of tumor cells from the lymphatic fluid into the general circulation via the thoracic duct and subclavian veins. In addition, shunts between lymphatic and blood vessels in lymph nodes may facilitate further haematogenous spread.

The term “angiogenesis”, as used herein refers to the sprouting of blood vessels from pre-existing blood vessels, characterized by endothelial cell proliferation and the proliferation and migration of tube forming cells. Angiogenesis can be triggered by certain pathological conditions, such as the growth of solid tumors and metastasis.

The terms “anti-angiogenic compound”, “anti-angiogenic factor”, “anti-angiogenic polypeptide” and “anti-angiogenic protein”, as used herein refer to a compound or factor that inhibits angiogenesis. i.e., the sprouting of blood vessels from pre-existing blood vessels, characterized by endothelial cell proliferation and the proliferation and migration of tube forming cells. It follows that anti-angiostatic activity means inhibition of angiogenesis.

The term “biologically active form”, as used herein relative to sVEGFR3 means any form of sVEGFR3 that exhibits an inhibitory effect on lymphangiogenesis, lymphatic metastasis or angiogenesis. The inhibitory effect on lymphangiogenesis, lymphatic metastasis or angiogenesis may be evaluated using any of a number of assays routinely employed by those of skill in the art. For angiogenesis, such assays include, but are not limited to, an endothelial cell migration assay, a Matrigel tube formation assay, endothelial and tumor cell proliferation assays, apoptosis assays and aortic ring assays, as further described below.

The term “exposing”, as used herein means bringing an anti-angiogenic factor-encoding vector in contact with a target cell. Such “exposing”, may take place in vitro, ex vivo or in vivo. The terms “complement” and “complementary” refer to two nucleotide sequences that comprise antiparallel nucleotide sequences capable of pairing with one another upon formation of hydrogen bonds between the complementary base residues in the antiparallel nucleotide sequences.

The term “native” refers to a gene or protein that is present in the genome of the wildtype virus or cell.

The term “naturally occurring” or “wildtype” is used to describe an object that can be found in nature as distinct from being artificially produced by man. For example, a protein or nucleotide sequence present in an organism (including a virus), which can be isolated from a source in nature and which has not been intentionally modified by man in the laboratory, is considered to be “naturally occurring”, “wildtype” or “native”.

The term “recombinant” as used herein with reference to nucleic acid molecules refers to a combination of nucleic acid molecules that are joined together using recombinant DNA technology into a progeny nucleic acid molecule. As used herein with reference to viruses, cells, and organisms, the terms “recombinant,” “transformed,” and “transgenic” refer to a host virus, cell, or organism into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Recombinant viruses, cells, and organisms are understood to encompass not only the end product of a transformation process, but also recombinant progeny thereof. A “non-transformed,” “non-transgenic,” or “non-recombinant” host refers to a wildtype virus, cell, or organism that does not contain the heterologous nucleic acid molecule.

The terms “transcriptional regulatory protein”, “transcriptional regulatory factor” and “transcription factor” are used interchangeably herein, and refer to a cytoplasmic or nuclear protein that binds a DNA response element and thereby transcriptionally regulates the expression of an associated gene or genes. Transcriptional regulatory proteins generally bind directly to a DNA response element, however in some cases binding to DNA may be indirect by way of binding to another protein that in turn binds to, or is bound to the DNA response element.

The term “promoter” refers to an untranslated DNA sequence usually located upstream of the coding region that contains the binding site for RNA polymerase II and initiates transcription of the DNA. The promoter region may also include other elements that act as regulators of gene expression. The term “minimal promoter” refers to a promoter element, particularly a TATA element that is inactive or has greatly reduced promoter activity in the absence of upstream activation elements.

The term “enhancer” within the meaning of the invention may be any genetic element, e.g., a nucleotide sequence that increases transcription of a coding sequence operatively linked to a promoter to an extent greater than the transcription activation effected by the promoter itself when operatively linked to the coding sequence, i.e. it increases transcription from the promoter.

A “termination signal sequence” within the meaning of the invention may be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site”, i.e. a site on a RNA transcript to which adenine residues will be added by post-transcriptional polyadenylation.

The term “homologous” as used herein with reference to a nucleic acid molecule refers to a nucleic acid sequence naturally associated with a host virus or cell.

As used herein, the term “sequence identity” means nucleic acid or amino acid sequence identity in two or more aligned sequences, when aligned using a sequence alignment program. The term “% homology” is used interchangeably herein with the term “% identity” herein and refers to the level of nucleic acid or amino acid sequence identity between two or more aligned sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J Mol. Biol. 215: 403-410 (1990), with software that is publicly available through the National Center for Biotechnology Information (http://www.ncbi.nlm.nih.gov/), or by visual inspection (see generally, Ausubel et al., infra). For purposes of the present invention, optimal alignment of sequences for comparison is most preferably conducted by the local homology algorithm of Smith & Waterman, Adv. Appl. Math. 2: 482 (1981). See, also, Altschul, S. F., et al., 1990 and Altschul, S. F. et al., 1997.

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the sequence comparison algorithms described herein, e.g. the Smith-Waterman algorithm, or by visual inspection.

In accordance with the present invention, also encompassed are sequence variants which encode self-processing cleavage polypeptides and polypeptides themselves that have 80, 85, 88, 89, 90, 91, 92, 93, 94, 95, 96, 97, 98, 99% or more sequence identity to the native or specified sequence.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe; while high stringency conditions are used to identify sequences having about 80% or more sequence identity with the probe.

Moderate and high stringency hybridization conditions are well known in the art (see, for example, Sambrook, et al, 1989, Chapters 9 and 11, and in Ausubel, F. M., et al., 1993. An example of high stringency conditions includes hybridization at about 42° C. in 50% formamide, 5×SSC, 5× Denhardt's solution, 0.5% SDS and 100 mg/ml denatured carrier DNA followed by washing two times in 2×SSC and 0.5% SDS at room temperature and two additional times in 0.1×SSC and 0.50% SDS at 42 [symbol] C. 2A sequence variants that encode a polypeptide with the same biological activity as the 2A polypeptides described herein and hybridize under moderate to high stringency hybridization conditions are considered to be within the scope of the present invention.

The terms “transcriptional regulatory protein”, “transcriptional regulatory factor” and “transcription factor” are used interchangeably herein, and refer to a cytoplasmic or nuclear protein that binds a DNA response element and thereby transcriptionally regulates the expression of an associated gene or genes. Transcriptional regulatory proteins generally bind directly to a DNA response element, however in some cases binding to DNA may be indirect by way of binding to another protein that in turn binds to, or is bound to the DNA response element.

As used herein, the terms “stably transformed”, “stably transfected” and “transgenic” refer to cells that have a non-native (heterologous) nucleic acid sequence integrated into the genome. Stable transformation is demonstrated by the establishment of cell lines or clones comprised of a population of daughter cells containing the transfecting DNA. In some cases, “transformation” is not stable, i.e., it is transient. In the case of transient transformation, the exogenous or heterologous DNA is expressed, however, the introduced sequence is not integrated into the genome.

The term “expression” refers to the transcription and/or translation of an endogenous gene, transgene or coding region in a cell. In the case of an antisense construct, expression may refer to the transcription of the antisense DNA only.

As used herein, the terms “biological activity” and “biologically active”, refer to the activity attributed to a particular protein in a cell line in culture or in vivo. It will be appreciated that the “biological activity” of such a protein may vary somewhat dependent upon in vitro or in vivo conditions and is generally reported as a range of activity. Accordingly, a “biologically inactive” form of a protein refers to a form of the protein that has been modified in a manner that interferes with the activity of the protein as it is found in nature.

As used herein, the terms “tumor,” “cancer” and “neoplasm” refer to a cell that exhibits a loss of growth control and forms unusually large clones of cells. Tumor or cancer cells generally have lost contact inhibition and may be invasive and/or have the ability to metastasize. Tumors or neoplasms include growths of cells in which the multiplication of the cells is uncontrolled and progressive. This is also referred to as neoplastic cell growth. Some such growths are benign, but others are malignant and may lead to death of the organism. Malignant neoplasms or cancers are distinguished from benign growths in that, in addition to exhibiting aggressive cellular proliferation, they may invade surrounding tissues and metastasize. Moreover, malignant neoplasms are characterized in that they show a greater loss of differentiation (greater “dedifferentiation”), and of their organization relative to one another and their surrounding tissues. This property is also called “anaplasia.”

Neoplasms treatable by the present invention include solid tumors, for example, carcinomas and sarcomas. Carcinomas include malignant neoplasms derived from epithelial cells which infiltrate, for example, invade, surrounding tissues and give rise to metastases. Adenocarcinomas are carcinomas derived from glandular tissue, or from tissues that form recognizable glandular structures. Another broad category of cancers includes sarcomas and fibrosarcomas, which are tumors whose cells are embedded in a fibrillar or homogeneous substance, such as embryonic connective tissue. The invention also provides methods of treatment of cancers of myeloid or lymphoid systems, including leukemias, lymphomas, and other cancers that typically are not present as a tumor mass, but are distributed in the vascular or lymphoreticular systems.

Further contemplated are methods for treatment of adult and pediatric oncology, growth of solid tumors/malignancies, myxoid and round cell carcinoma, locally advanced tumors, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer (including small cell carcinoma of the lungs, cutaneous T cell lymphoma, Hodgkin's lymphoma, non-Hodgkin's lymphoma, cancer of the adrenal cortex, ACTH-producing tumors, non-small cell lung cancers, breast cancer, including small cell carcinoma and ductal carcinoma), gastro-intestinal cancers (including stomach cancer, colon cancer, colorectal cancer, and polyps associated with colorectal neoplasia), pancreatic cancer, liver cancer, urological cancers (including bladder cancer, such as primary superficial bladder tumors, invasive transitional cell carcinoma of the bladder, and muscle-invasive bladder cancer), prostate cancer, malignancies of the female genital tract (including ovarian carcinoma, primary peritoneal epithelial neoplasms, cervical carcinoma, uterine endometrial cancers, vaginal cancer, cancer of the vulva, uterine cancer and solid tumors in the ovarian follicle), malignancies of the male genital tract (including testicular cancer and penile cancer), kidney cancer (including renal cell carcinoma, brain cancer (including intrinsic brain tumors, neuroblastoma, astrocytic brain tumors, gliomas, and metastatic tumor cell invasion in the central nervous system), bone cancers (including osteomas and osteosarcomas), skin cancers (including malignant melanoma, tumor progression of human skin keratinocytes, basal cell carcinoma, and squamous cell cancer), thyroid cancer, retinoblastoma, neuroblastoma, peritoneal effusion, malignant pleural effusion, mesothelioma, Wilms's tumors, gall bladder cancer, trophoblastic neo-plasms, hemangiopericytoma, and Kaposi's sarcoma.

The term “administering”, as used herein refers to delivering a composition, such as a composition containing a gene therapy vector encoding a soluble form of VEGFR3 to the cells of a subject. Such administering may take place in vivo, in vitro or ex vivo.

As used herein, “effective amount” relative to a vector encoding a soluble form of VEGFR3 refers to the vector administered to a mammalian subject, either as a single dose or as part of a series of doses and which is effective to result in an improved therapeutic outcome of the subject under treatment.

As used herein “treatment” of an individual or a cell is any type of intervention used in an attempt to alter the natural course of the individual or cell. Treatment includes, but is not limited to, administration of e.g., a vector encoding a soluble form of VEGFR3, a pharmaceutical composition, alone or in combination with other treatment modalities generally known in the art for treatment of cancer.

As used herein, the term “improved therapeutic outcome” relative to a cancer patient refers to a slowing or diminution of the growth of cancer cells or a solid tumor, a reduction in the total number of cancer cells or total tumor burden, or a decrease in or amelioration of adverse symptoms. Other indicia of improved therapeutic outcome include increased survival time and/or reduced side effects.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. In addition, compositions for specific administration to the CNS are included such as artificial cerebrospinal fluid (see Cunningham et al 2000, Cell Transplantation, Vol. 9, 585-594). Compositions comprising such carriers are formulated by well known conventional methods (see, for example, Remington's Pharmaceutical Sciences, 18th edition, A. Gennaro, ed., Mack Publishing Co., Easton, Pa., 1990).

As used herein, “subject” refers to the recipient of the therapy to be practiced according to the invention. The subject can be any animal, including a vertebrate, but will preferably be a mammal. If a mammal, the subject will preferably be a human, but may also be a domestic livestock, laboratory subject or pet animal.

Soluble Vascular Endothelial Growth Factor 3 (sVEGFR3)

Vascular endothelial growth factor (VEGF) is produced when tumors grow to a certain size (2-3 mm) and become hypoxic, or oxygen starved. VEGF participates in a signaling process that triggers the activation, division, and migration of endothelial cells that line blood vessel walls, resulting in the growth of capillaries from the blood vessels and into the VEGF-secreting tumor. (See, e.g., Ferrara, N., 1999, Curr. Top. Microbiol. Immunol. 237, 1-30; Risau, W., 1997, Nature 386, 671-674; and Yancopoulus, G. D. et al., 2000, Nature 407, 242-248.)

The vascular endothelial growth factor (VEGF) receptor family in mammals contains three members, VEGFR1(Flt-1), VEGFR2(KDR/Flk-1) and VEGFR3 (Flt-4), which directly regulate the formation of blood vessels and lymphatic vessels. FLT4 is a receptor tyrosine kinase closely related in structure to the products of the VEGFR-1 and VEGFR-2 genes. The mature form of FLT4 differs from the VEGFR-1 and VEGFR-2 in that it is proteolytically cleaved in the extracellular domain into two disulfide-linked polypeptides of 4.5 and 5.8 kb. The FLT4 gene encodes mRNAs which exhibit alternative 3′ exons. VEGFR3 (Flt-4) and its use as a diagnostic tool in studying emerging and/or existing neoplasms is described in U.S. Pat. No. 5,776,755, expressly incorporated by reference herein.

A number of clinical and experimental studies suggest that tumor-induced lymphangiogenesis driven by vascular endothelial growth factor (VEGF)-C- and/or VEGF-D-induced activation of VEGF receptor (VEGFR)-3 may promote metastasis to regional lymph nodes. See, e.g., Gershenwald J E and Fidler I J Science 296(5574):1811-2, 2002. Pepper M S et al., Cell Tissue Res. 314(1):167-77, 2003 and Cassella M and Skobe M, Ann N Y Acad Sci. 979:120-30, 2001. VEGF-C overexpression in breast cancer cells was shown to significantly increase intratumoral lymphangiogenesis, resulting in enhanced metastasis to regional lymph nodes and degree of tumor lymphangiogenesis appears to correlate with the extent of lymph node and lung metastases (Skobe M et al, Nat Med. 7(2):192-8, 2001). These results established the occurrence and biological significance of lymphangiogenesis in cancer and identified VEGF-C as an essential inducer of tumor lymphangiogenesis and metastasis. Furthermore, suppression of VEGF-C with neutralizing antibodies or block of the VEGFR3 signaling pathways by a soluble VEGFR3-Ig fusion protein in tumor-bearing mice has been shown to suppress tumor lymphangiogenesis, thereby resulting in the inhibition of tumor metastasis to regional lymph nodes (Karpanen et al Cancer Res. 61(5):1786-90, 2001; Makinen et al, Nat Med. 7(2)-199-205, 2001; He Y et al, J Natl Cancer Inst. 94(11):819-25, 2002). An AAV vector expressing a VEGFR-3-specific mutant form of VEGF-C (VEGF-C156S) was shown to potently induce lymphangiogenesis in transgenic mouse embryos and functional cutaneous lymphatic vessels were detected after long-term expression of VEGF-C156S (Saaristo A, et al., J Exp Med. 196(6):719-30, 2002).

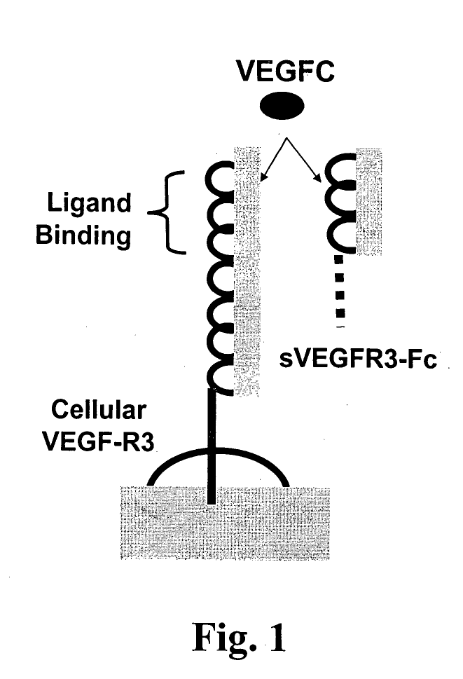

The present invention provides gene therapy vectors that include the coding sequence for a biologically active form of soluble VEGFR3 (which includes the ligand-binding domains of the VEGFR3 receptor was fused to human IgG Fc), presented herein as SEQ ID NO: 1 with the corresponding translated amino acid sequence presented herein as SEQ ID NO:2. A schematic depiction of sVEGFR3 showing the ligand-binding domains of the VEGFR3 receptor fused to human IgG Fc is presented in FIG. 1.

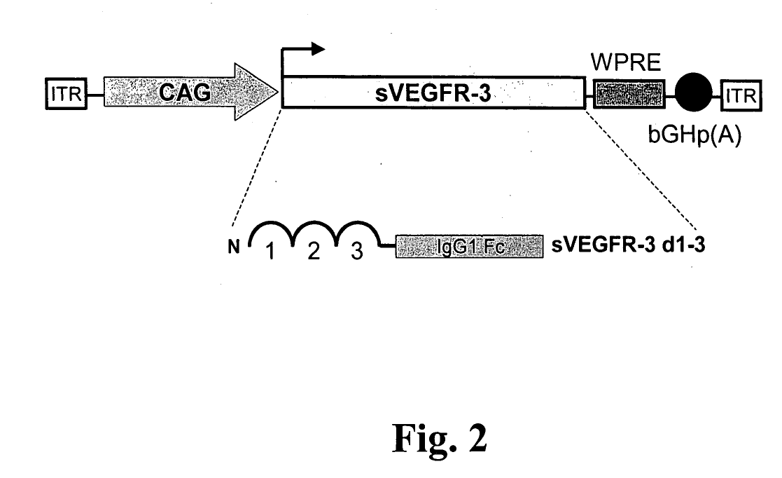

In order to evaluate the effects of soluble VEGFR3, a chimeric molecule designated sVEGFR-3Fc, and encompassing the 1-3 Ig-like domains of human VEGFR-3 gene fused to the human IgG1 heavy chain (Fc) was cloned into an AAV vector downstream of the constitutive CAG promoter and upstream of the Woodchuck Hepatitis Virus Post-transcriptional regulatory element (WPRE) and bovine growth hormone polyadenylation sequence (bGHpA). ITRs represent the AAV-2 inverted terminal repeats. A schematic depiction of this exemplary AAV vector which encodes a soluble form of VEGFR3 (rAAV-VEGFR-3Fc) is provided in FIG. 2. Thus, in one embodiment, the present invention provides an exemplary AAV vector comprising the coding sequence set forth in SEQ ID NO: 6. In accordance with the present invention, also encompassed are sequences that exhibit at least 80, 85, 88, 89, 90, 91, 92, 93, 94, 95, 96, 97, 98, 99% or more sequence identity to the exemplary AAV vector sequence set forth in SEQ ID NO: 6.

Sequence variants include nucleic acid molecules that encode sVEGFR3 as is encoded by the sVEGFR3 sequence described herein. Thus, where the coding frame of a soluble form of VEGFR3 is known, it will be appreciated that as a result of the degeneracy of the genetic code, a number of coding sequences can be produced. For example, the triplet CGT encodes the amino acid arginine. Arginine is alternatively encoded by CGA, CGC, CGG, AGA, and AGG. Therefore it is appreciated that such substitutions in the coding region fall within the sequence variants that are covered by the present invention.

It is further appreciated that such sequence variants may or may not hybridize to the parent sequence under conditions of high stringency. This would be possible, for example, when the sequence variant includes a different codon for each of the amino acids encoded by the parent nucleotide. Such variants are, nonetheless, specifically contemplated and encompassed by the present invention.

In accordance with the present invention, also encompassed are sequences that exhibit at least 80, 85, 88, 89, 90, 91, 92, 93, 94, 95, 96, 97, 98, 99% or more sequence identity to the sVEGFR3 sequence described herein.

Gene Delivery Vectors

The present invention contemplates the use of any vector for introduction of one or more genes encoding a soluble form of VEGFR3 into mammalian cells. Exemplary vectors include but are not limited to, viral and non-viral vectors, such as' retroviruses (including lentiviruses), adenovirus (Ad) vectors including replication competent, replication deficient and gutless forms thereof, adeno-associated virus (AAV) vectors, simian virus 40 (SV-40) vectors, bovine papilloma virus vectors, Epstein-Barr virus vectors, herpes virus vectors, vaccinia virus vectors, Moloney murine leukemia virus vectors, Harvey murine sarcoma virus vectors, murine mammary tumor virus vectors, Rous sarcoma virus vectors and nonviral plasmid vectors. In one preferred approach, the vector is a viral vector. Viruses can efficiently transduce cells and introduce their own DNA into a host cell. In generating recombinant viral vectors, non-essential genes are replaced with a gene or coding sequence for sVEGFR3.

In constructing viral vectors, non-essential genes are replaced with one or more genes encoding a soluble form of VEGFR3. Typically, the vector comprises an origin of replication and the vector may or may not also comprise a “marker” or “selectable marker” function by which the vector can be identified and selected. While any selectable marker can be used, selectable markers for use in such expression vectors are generally known in the art and the choice of the proper selectable marker will depend on the host cell. Examples of selectable marker genes which encode proteins that confer resistance to antibiotics or other toxins include ampicillin, methotrexate, tetracycline, neomycin (Southern et al., J., J Mol Appl Genet. 1982;1(4):327-41 (1982)), mycophenolic acid (Mulligan et al., Science 209:1422-7 (1980)), puromycin, zeomycin, hygromycin (Sugden et al., Mol Cell Biol. 5(2):410-3 (1985)) or G418.

Reference to a vector or other DNA sequences as “recombinant” merely acknowledges the operable linkage of DNA sequences which are not typically operably linked as isolated from or found in nature. Regulatory (expression/control) sequences are operatively linked to a nucleic acid coding sequence when the expression/control sequences regulate the transcription and, as appropriate, translation of the nucleic acid sequence. Thus expression/control sequences can include promoters, enhancers, transcription terminators, a start codon (i.e., ATG) in front of the coding sequined, splicing signal for introns and stop codons.

Adenovirus gene therapy vectors are known to exhibit strong expression in vitro, excellent titer, and the ability to transduce dividing and non-dividing cells in vivo (Hitt et al., Adv in Virus Res 55:479-505 (2000)). When used in vivo these vectors lead to strong but transient gene expression due to immune responses elicited to the vector backbone. The recombinant Ad vectors for use in the instant invention comprise: (1) a packaging site enabling the vector to be incorporated into replication-defective Ad virions; and (2) a therapeutic compound coding sequence. Other elements necessary or helpful for incorporation into infectious virions, include the 5′ and 3′ Ad ITRs, the E2 and E3 genes, etc.

Replication-defective Ad virions encapsulating the recombinant Ad vectors of the instant invention are made by standard techniques known in the art using Ad packaging cells and packaging technology. Examples of these methods may be found, for example, in U.S. Pat. No. 5,872,005, incorporated herein by reference in its entirety. A sequence encoding a soluble form of VEGFR3 is commonly inserted into an adenovirus in the deleted E1A, E1B or E3 region of the virus genome. Preferred adenoviral vectors for use in practicing the invention do not express one or more wild-type Ad gene products, e.g., E1a, E1b, E2, E3, E4. Preferred embodiments are virions that are typically used together with packaging cell lines that complement the functions of E1, E2A, E4 and optionally the E3 gene regions. See, e.g. U.S. Pat. Nos. 5,872,005, 5,994,106, 6,133,028 and 6,127,175, expressly incorporated by reference herein in their entirety. Adenovirus vectors are purified and formulated using standard techniques known in the art.

Recombinant AAV vectors are characterized in that they are capable of directing the expression and the production of the selected transgenic products in targeted cells. Thus, the recombinant vectors comprise at least all of the sequences of AAV essential for encapsidation and the physical structures for infection of target cells.

Recombinant AAV (rAAV) virions for use in practicing the present invention may be produced using standard methodology, known to those of skill in the art and are constructed such that they include, as operatively linked components in the direction of transcription, control sequences including transcriptional initiation and termination sequences, and the coding sequence for a soluble form of VEGFR3. These components are bounded on the 5′ and 3′ end by functional AAV ITR sequences. By “functional AAV ITR sequences” is meant that the ITR sequences function as intended for the rescue, replication and packaging of the AAV virion. Hence, AAV ITRs for use in the vectors of the invention need not have a wild-type nucleotide sequence, and may be altered by the insertion, deletion or substitution of nucleotides or the AAV ITRs may be derived from any of several AAV serotypes. An AAV vector is a vector derived from an adeno-associated virus serotype, including without limitation, AAV-1, AAV-2, AAV-3, AAV-4, AAV-5, AAV-6, AAV-7, AAV-8, etc. Preferred AAV vectors have the wild type REP and CAP genes deleted in whole or part, but retain functional flanking ITR sequences. Table 1 illustrates exemplary AAV serotypes for use in gene transfer.

TABLE 1
AAV Serotypes For Use In Gene Transfer.

| | | Genome Size | Homology vs | Immunity in |
|---|---|---|---|---|
| Serotype | Origin | (bp) | AAV2 | Human Population |
| AAV-1 | Human Specimen | 4718 | NT: 80% | NAB: 20% |
| | | | AA: 83% | |
| AAV-2 | Human Genital | 4681 | NT: 100% | NAB: 27-53% |
| | Abortion tissue | | AA: 100% | |
| | Amnion Fluid | | | |
| AAV-3 | Human Adenovirus | 4726 | NT: 82% | cross reactivity with AAV2 |
| | Specimen | | AA: 88% | NAB |
| AAV-4 | African Green Monkey | 4774 | NT: 66% | Unknown |
| | | | AA: 60% | |
| AAV-5 | Human Genital Lesion | 4625 | NT: 65% | ELISA: 45% NAB: 0% |
| | | | AA: 56% | |
| AAV-6 | Laboratory isolate | 4683 | NT: 80% | 20% |
| | | | AA: 83% | |
| AAV-7 | Isolated from Heart | 4721 | NT: 78% | NAB: <1:20 (˜5%) |
| | DNA of Rhesus | | AA: 82% | |
| | Monkey | | | |
| AAV-8 | Isolated from Heart | 4393 | NT: 79% | NAB: <1:20 (˜5%) |
| | DNA of Rhesus | | AA: 83% | |
| | Monkey | | | |

Typically, an AAV expression vector is introduced into a producer cell, followed by introduction of an AAV helper constrict, where the helper construct includes AAV coding regions capable of being expressed in the producer cell and which complement AAV helper functions absent in the AAV vector. The helper construct may be designed to down regulate the expression of the large REP proteins (Rep78 and Rep68), typically by mutating the start codon following p5 from ATG to ACG, as described in U.S. Pat. No. 6,548,286, expressly incorporated by reference herein. This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions encapsulating the recombinant AAV vectors of the instant invention are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, expressly incorporated by reference herein in their entirety. Further compositions and methods for packaging are described in Wang et al. (US 2002/0168342), also incorporated by reference herein in its entirety, and include those techniques within the knowledge of those of skill in the art.

Approximately 40 serotypes of AAV are currently known, however, new serotypes and variants of existing serotypes are still being identified today and are considered within the scope of the present invention. See Gao et al (2002), PNAS 99(18):11854-6; Gao et al (2003), PNAS 100(10):6081-6; Bossis and Chiorini (2003), J. Virol. 77(12):6799-810). Different AAV serotypes are used to optimize transduction of particular target cells or to target specific cell types within a particular target tissue, such as the brain. The use of different AAV serotypes may facilitate targeting of malignant tissue. AAV serotypes including 1, 2, 4, 5 and 6 have been shown to transduce brain tissue. See, e.g., Davidson et al (2000), PNAS 97(7)3428-32; Passini et al (2003), J. Virol 77(12):7034-40). Particular AAV serotypes may more efficiently target and/or replicate in target tissue or cells. A single self-complementary AAV vector can be used in practicing the invention in order to increase transduction efficiency and result in faster onset of transgene expression (McCarty et al., Gene Ther. 2001 Aug.;8(16):1248-54).

In practicing the invention, host cells for producing rAAV virions include mammalian cells, insect cells, microorganisms and yeast. Host cells can also be packaging cells in which the AAV REP and CAP genes are stably maintained in the host cell or alternatively host cells can be producer cells in which the AAV vector genome is stably maintained. Exemplary packaging and producer cells are derived from 293, A549 or HeLa cells. AAV vectors are purified and formulated using standard techniques known in the art.

Retroviral vectors are a common tool for gene delivery (Miller, 1992, Nature 357: 455-460). Retroviral vectors and more particularly lentiviral vectors may be used in practicing the present invention. Retroviral vectors have been tested and found to be suitable delivery vehicles for the stable introduction of a variety of genes of interest into the genomic DNA of a broad range of target cells. The ability of retroviral vectors to deliver unrearranged, single copy transgenes into cells makes retroviral vectors well suited for transferring genes into cells. Further, retroviruses enter host cells by the binding of retroviral envelope glycoproteins to specific cell surface receptors on the host cells. Consequently, pseudotyped retroviral vectors in which the encoded native envelope protein is replaced by a heterologous envelope protein that has a different cellular specificity than the native envelope protein (e.g., binds to a different cell-surface receptor as compared to the native envelope protein) may also find utility in practicing the present invention. The ability to direct the delivery of retroviral vectors encoding a transgene to a specific type of target cells is highly desirable for gene therapy applications.

The present invention provides retroviral vectors which include e.g., retroviral transfer vectors comprising one or more transgene sequences and retroviral packaging vectors comprising one or more packaging elements. In particular, the present invention provides pseudotyped retroviral vectors encoding a heterologous or functionally modified envelope protein for producing pseudotyped retrovirus.

The core sequence of the retroviral vectors of the present invention may be readily derived from a wide variety of retroviruses, including for example, B, C, and D type retroviruses as well as spumaviruses and lentiviruses (see RNA Tumor Viruses, Second Edition, Cold Spring Harbor Laboratory, 1985). An example of a retrovirus suitable for use in the compositions and methods of the present invention includes, but is not limited to, lentivirus. Other retroviruses suitable for use in the compositions and methods of the present invention include, but are not limited to, Avian Leukosis Virus, Bovine Leukemia Virus, Murine Leukemia Virus, Mink-Cell Focus-Inducing Virus, Murine Sarcoma Virus, Reticuloendotheliosis virus and Rous Sarcoma Virus. Particularly preferred Murine Leukemia Viruses include 4070A and 1504A (Hartley and Rowe, J. Virol. 19:19-25, 1976), Abelson (ATCC No. VR-999), Friend (ATCC No. VR-245), Graffi, Gross (ATCC No. VR-590), Kirsten, Harvey Sarcoma Virus and Rauscher (ATCC No. VR-998), and Moloney Murine Leukemia Virus (ATCC No. VR-190). Such retroviruses may be readily obtained from depositories or collections such as the American Type Culture Collection (“ATCC”; Rockville, Md.), or isolated from known sources using commonly available techniques.

Preferably, a retroviral vector sequence of the present invention is derived from a lentivirus. A preferred lentivirus is a human immunodeficiency virus, e.g., type 1 or 2 (i.e., HIV-1 or HIV-2, wherein HIV-1 was formerly called lymphadenopathy associated virus 3 (HTLV-III) and acquired immune deficiency syndrome (AIDS)-related virus (ARV)), or another virus related to HIV-1 or HIV-2 that has been identified and associated with AIDS or AIDS-like disease. Other lentivirus vectors include, a sheep Visna/maedi virus, a feline immunodeficiency virus (FIV), a bovine lentivirus, simian immunodeficiency virus (SIV), an equine infectious anemia virus (EIAV), and a caprine arthritis-encephalitis virus (CAEV).

The various genera and strains of retroviruses suitable for use in the compositions and methods are well known in the art (see, e.g., Fields Virology, Third Edition, edited by B. N. Fields et al., Lippincott-Raven Publishers (1996), see e.g., Chapter 58, Retroviridae: The Viruses and Their Replication, Classification, pages 1768-1771, including Table 1, incorporated herein by reference).

The present invention provides retroviral packaging systems for generating producer cells and producer cell lines that produce retroviruses, and methods of making such packaging systems. Accordingly, the present invention also provides producer cells and cell lines generated by introducing a retroviral transfer vector into such packaging systems (e.g., by transfection or infection), and methods of making such packaging cells and cell lines.

The retroviral packaging systems for use in practicing the present invention comprise at least two packaging vectors: a first packaging vector which comprises a first nucleotide sequence comprising a gag, a pol, or gag and pol genes; and a second packaging vector which comprises a second nucleotide sequence comprising a heterologous or functionally modified envelope gene. In a preferred embodiment, the retroviral elements are derived from a lentivirus, such as HIV. Preferably, the vectors lack a functional tat gene and/or functional accessory genes (vif, vpr, vpu, vpx, nef). In another preferred embodiment, the system further comprises a third packaging vector that comprises a nucleotide sequence comprising a rev gene. The packaging system can be provided in the form of a packaging cell that contains the first, second, and, optionally, third nucleotide sequences.

The invention is applicable to a variety of retroviral systems, and those skilled in the art will appreciate the common elements shared across differing groups of retroviruses. The description herein uses lentiviral systems as a representative example. However, all retroviruses share the features of enveloped virions with surface projections and containing one molecule of linear, positive-sense single stranded RNA, a genome consisting of a dimer, and the common proteins gag, pol and env.

Lentiviruses share several structural virion proteins in common, including the envelope glycoproteins SU (gp120) and TM (gp41), which are encoded by the env gene; CA (p24), MA (p 17) and NC (p7-11), which are encoded by the gag gene; and RT, PR and IN encoded by the pol gene. HIV-1 and HIV-2 contain accessory and other proteins involved in regulation of synthesis and processing virus RNA and other replicative functions. The accessory proteins, encoded by the vif, vpr, vpu/vpx, and nef genes, can be omitted (or inactivated) from the recombinant system. In addition, tat and rev can be omitted or inactivated, e.g., by mutation or deletion.

First generation lentiviral vector packaging systems provide separate packaging constructs for gag/pol and env, and typically employ a heterologous or functionally modified envelope protein for safety reasons. In second generation lentiviral vector systems, the accessory genes, vif, vpr, vpu and nef, are deleted or inactivated. Third generation lentiviral vector systems are those from which the tat gene has been deleted or otherwise inactivated (e.g., via mutation).

Compensation for the regulation of transcription normally provided by tat can be provided by the use of a strong constitutive promoter, such as the human cytomegalovirus immediate early (HCMV-IE) enhancer/promoter. Other promoters/enhancers can be selected based on strength of constitutive promoter activity, specificity for target tissue (e.g., liver-specific promoter), or other factors relating to desired control over expression, as is understood in the art. For example, in some embodiments, it is desirable to employ an inducible promoter such as tet to achieve controlled expression. The gene encoding rev is preferably provided on a separate expression construct, such that a typical third generation lentiviral vector system will involve four plasmids: one each for gagpol, rev, envelope and the transfer vector. Regardless of the generation of packaging system employed, gag and pol can be provided on a single construct or on separate constructs.

Typically, the packaging vectors are included in a packaging cell, and are introduced into the cell via transfection, transduction or infection. Methods for transfection, transduction or infection are well known by those of skill in the art. A retroviral transfer vector of the present invention can be introduced into a packaging cell line, via transfection, transduction or infection, to generate a producer cell or cell line. The packaging vectors of the present invention can be introduced into human cells or cell lines by standard methods including, e.g., calcium phosphate transfection, lipofection or electroporation. In some embodiments, the packaging vectors are introduced into the cells together with a dominant selectable marker, such as neo, DHFR, Gln synthetase or ADA, followed by selection in the presence of the appropriate drug and isolation of clones. A selectable marker gene can be linked physically to genes encoding by the packaging vector.

Stable cell lines, wherein the packaging functions are configured to be expressed by a suitable packaging cell, are known. For example, see U.S. Pat. No. 5,686,279; and Ory et al., Proc. Natl. Acad. Sci. (1996) 93:11400-11406, which describe packaging cells. Further description of stable cell line production can be found in Dull et al., 1998, J. Virology 72(11):8463-8471; and in Zufferey et al., 1998, J. Virology 72(12):9873-9880

Zufferey et al., 1997, Nature Biotechnology 15:871-875, teach a lentiviral packaging plasmid wherein sequences 3′ of pol including the HIV-1 envelope gene are deleted. The construct contains tat and rev sequences and the 3′ LTR is replaced with poly A sequences. The 5′ LTR and psi sequences are replaced by another promoter, such as one which is inducible. For example, a CMV promoter or derivative thereof can be used.

The packaging vectors of interest may contain additional changes to the packaging functions to enhance lentiviral protein expression and to enhance safety. For example, all of the HIV sequences upstream of gag can be removed. Also, sequences downstream of envelope can be removed. Moreover, steps can be taken to modify the vector to enhance the splicing and translation of the RNA.

Optionally, a conditional packaging system is used, such as that described by Dull et al., 1998, J. Virology 72(11):8463-8471. Also preferred is the use of a self-inactivating vector (SIN), which improves the biosafety of the vector by deletion of the HIV-1 long terminal repeat (LTR) as described, for example, by Zufferey et al., 1998, J. Virology 72(12):9873-9880. Inducible vectors can also be used, such as through a tet-inducible LTR.

Regulatory Elements

The gene therapy vectors of the invention typically include heterologous control sequences, which include, but are not limited to tumor selective promoters and enhancers, including but not limited to the E2F promoter and the telomerase (hTERT) promoter; the cytomegalovirus enhancer/chicken beta-actin/Rabbit β-globin promoter (CAG promoter; Niwa H. et al. 1991. Gene 108(2):193-9; Garg et al., J. Immunol., 173:550-558, 2004; ATCC SCRC-1033); the elongation factor 1-alpha promoter (EF1-alpha) promoter (Kim D W et al. 1990. Gene. 91(2):217-23 and Guo Z S et al. 1996. Gene Ther. 3(9):802-10); a glial specific promoter (e.g. glial fibrary acid protein promoter) and a neuron specific promoter (e.g. neuron specific enolase promoter or synapsin promoter).

In some cases constitutive promoters, such as the cytomegalovirus (CMV) immediate early promoter, the RSV LTR, the MoMLV LTR, the CAG promoter, the phosphoglycerate kinase-1 promoter (PGK) or the SV-40 promoter may be employed. Exemplary vectors employing CMV immediate early promoter and the CAG promoter include expression vector pCLGFPA (Genbank Accession No. AJ575208) and cloning vector pTurbo-Cre (Genbank Accession No. AF334827).

The gene therapy vectors of the invention may also include enhancers and coding sequences for signal peptides. The vector constructs may or may not include an intron. Neural or glial specific promoters may be included in the vectors of the invention as a means to limit expression to specific cell types in the brain. Thus it will be appreciated that gene therapy vectors of the invention may include any of a number of transgenes, combinations of transgenes and transgene/regulatory element combinations.

Exemplary gene regulation systems that may be employed in practicing the present invention include, the Drosophila ecdysone system (Yao and Evans, 1996, Proc. Nat. Acad. Sci.; 93:3346), the Bombyx ecdysone system (Suhr et al., 1998, Proc. Nat. Acad. Sci., 95:7999), the Valentis GeneSwitch® synthetic progesterone receptor system which employs RU-486 as the inducer (Osterwalder et al., 2001, Proc Natl Acad Sci 98(22):12596-601); the Tet™ & RevTet™ Systems (BD Biosciences Clontech), which employs small molecules, such as tetracycline (Tc) or analogues, e.g. doxycycline, to regulate (turn on or off) transcription of the target (Knott A et al., Biotechniques 2002, 32(4):796, 798, 800); ARIAD Regulation Technology which is based on the use of a small molecule to bring together two intracellular molecules, each of which is linked to either a transcriptional activator or a DNA binding protein. When these components come together, transcription of the gene of interest is activated. Ariad has two major systems: a system based on homodimerization and a system based on heterodimerization (Rivera et al., 1996, Nature Med, 2(9):1028-1032; Ye et al., 2000, Science 283: 88-91)

Preferred gene regulation systems for use in practicing the present invention are the ARIAD Regulation Technology and the Tet™ & RevTet™ Systems.

The gene therapy vectors and constructs described above may be introduced into cells using standard methodology known in the art. Such techniques include transfection using calcium phosphate, micro-injection into cultured cells (Capecchi, Cell 22:479-488 [1980]), electroporation (Shigekawa et al., BioTechn., 6:742-751 [1988]), liposome-mediated gene transfer (Mannino et al., BioTechn., 6:682-690 [1988]), lipid-mediated transduction (Felgner et al., Proc. Natl. Acad. Sci. USA 84:7413-7417 [1987]), and nucleic acid delivery using high-velocity microprojectiles (Klein et al., Nature 327:70-73 [1987]).

In Vitro Evaluation Of Angiogenesis

The effectiveness of a given vector encoding sVEGFR3 may be evaluated in vitro using any of a number of methods known in the art. Exemplary in vitro angiogenesis assays include, but are not limited to, an endothelial cell migration assay, a Matrigel tube formation assay, endothelial and tumor cell proliferation assays, apoptosis assays and aortic ring assays.

The rate of endothelial cell migration was evaluated using human umbilical vein endothelial cells (HUVEC) using a modified Boyden chamber assay as described by Clyman et al., 1994, Cell Adhes Commun. 1(4):333-42 and Lin, P et al., 1998, Cell Growth Differ. 9(1):49-58.

In Vivo Evaluation Of Angiogenesis

In vivo gene expression as well as the effectiveness of a given vector encoding sVEGFR3 may be evaluated in vivo using any of a number of methods known in the art. For example, gene expression may be evaluated by measurement of the amount of sVEGFR3 in the serum of animals following administration of a vector encoding sVEGFR3, e.g., by immunoassays, such as ELISA (as further described below), competitive immunoassays, radioimmunoassays, Western blot, indirect immunofluorescent assays and the like. The activity, expression and/or production of mRNA for sVEGFR3 may also be determined by Northern blot and/or reverse transcriptase polymerase chain reaction (RT-PCR).

Exemplary in vivo angiogenesis models include, but are not limited to, in a B16 B1/6 Mouse melanoma metastasis model (described below); a B16F10-luc metastasis model with Xenogen Imaging (described below); a Lewis Lung Carcinoma (LLC) Xenograft Resection Model (O'Reilly et al, 1994, Cell. 79(2):315-28); a LLC-luc metastasis model/Xenogen Imaging; a LLC-luc SC resection model/Xenogen Imaging; a RIP-Tag pancreatic islet carcinoma transgenic model (Hanahan et al., Nature, 315(6015):115-122, 1985 and Bergers et al., Science, 284:808-811, 1999); an orthotopic breast cancer model MDA-231 (Hiraga T. et al., 2001, Cancer Res. 61(11):4418-24); a C6 glioma model (Griscelli F, et al., 1998, Proc Natl Acad Sci USA. 95(11):6367-72) an LnCP prostate cancer model (Horoszewicz J S et al., Cancer Res. 43(4):1809-18, 1983); and a PC-3 Xenograft pancreatic tumor model (Donaldson J T et al., 1990, Int J Cancer. 46(2):238-44).

The RIP-Tag spontaneous pancreatic islet carcinoma model makes use of transgenic mice which have been genetically modified to express a rat insulin promoter (RIP) driven simian virus 40 (SV-40) antigen and develop islet cell carcinomas as a result of SV-40 oncogene expression in pancreatic islet cells. In the model, tumor development proceeds through a series of well-defined stages.

In Vitro Evaluation of Lymphangiogenesis and Lymphatic Metastasis

The effectiveness of a given vector encoding sVEGFR3 may be evaluated in vitro using any of a number of methods known in the art. Many in vitro assays to test for modulators of lymphangiogenesis are similar to those used to evaluate angiogenesis. For example, in vitro lymphangiogenesis assays may include, but are not limited to, lymphatic endothelial cell proliferation assays, lymphatic endothelial migration assays, and assays for the formation of lymphatic capillaries in response to pro-lymphangiogenic factors in vitro and ex vivo. Other assays may include testing the ability of sVEGFR3 to block the biochemical and biological activities of pro-lymphangiogenic growth factor signaling pathways in responsive cells. For example, the ability of sVEGFR3 to inhibit the lymphangiogenic growth factor, VEGF-C or VEGF-D, may be tested in responsive tissue culture cells which have been engineered to be mitogenic in response to VEGF-C stimulation.

In Vivo Evaluation of Lymphangiogenesis and Lymphatic Metastasis

The ability of sVEGFR3 to block lymphatic-mediated metastasis can be evaluated in animal models which have been developed for tumors that are dependent on lymphangiogenesis for their growth and spread. Exemplary models are described in the Examples, below, and may include, but are not limited to, metastatic models of prostate, melanoma, breast, head & neck, and renal cell carcinomas. Tumor variant cell lines that preferentially metastasize to lymph nodes may be selected or tumor lines that highly express VEGF-C or VEGF-D may be used for development of animal tumor models for lymphatic metastases.

Therapeutic Applications of the Current Invention

The invention contemplates administration of the recombinant vectors described herein to a patient with a tumor in order to slow or halt completely the growth of the tumor. Administration to the patient may be by any known method, including both in vivo and ex vivo modes of administration.

In vivo delivery involves delivery of a gene therapy vectors of the invention directly to a patient. In some cases, the vector is delivered to a depot organ, e.g., liver or muscle, by intraportal (IP) or intramuscular (IM) injection, respectively. In other approaches, the vector is delivered intravenously (IV). Such delivery may also be by the intraperitoneal route or by delivery directly to the tumor site (intratumoral or IT). Non-invasive methods, such as oral delivery, are also contemplated. In some cases, delivery may be accomplished by an ex vivo route. Ex vivo delivery involves ex vivo (outside the body) transduction of cells by the recombinant vectors, followed by administration of the transduced cells to the patient.

The gene therapy vectors of the invention are delivered in an amount effective yield to a therapeutic level of sVEGFR3 encoded by the vector in the vicinity of cancer cells or a tumor. Sustained or continuous expression of sVEGFR3 in the generalized circulation or surrounding the tumor site is achieved following delivery of the gene therapy vectors in vivo. The therapeutic dose of serum sVEGFR3 required to block the effector targets (i.e. VEGF-C or VEGF-D) involved in lymphangiogenesis or angiogenesis is dependent on the nature and aggressiveness of the tumor. For example, in preclinical metastatic animal models of prostate and melanoma it is preferred that sVEGFR3 be present in the serum at a level of about 10-20 μg/ml. The level of sVEGFR3 expression following gene delivery will be dependent on individual tumor types and on its dependency for lymphangiogenesis for metastatic spread and growth. For therapeutic efficacy, the vector is administered such that an improved therapeutic outcome results.

The present invention contemplates treatment regimens that include the use of gene therapy vectors that encode sVEGFR3, alone or in combination with any of a number of modes of therapeutic intervention typically employed by those of skill in the art to treat cancer. In general standard therapeutic regimens for cancer treatment, including surgery, chemotherapy and radiation therapy suffer from suffer from a number of deficiencies the most important of which are a lack of efficacy and frequent toxic side effects. Chemotherapeutic agents for use in practicing the invention include any of a number of agents with established use in cancer therapy. More recently immunotherapy methods and treatments that involve direct administration of anti-angiogenic compounds or agents are under development for cancer treatment. There remains a serious need for specific, less toxic cancer therapies.

Accordingly, the present invention includes improved cancer treatment regimens that involve the use of gene therapy vectors that encode sVEGFR3 in combination with one or more of chemotherapy, radiation therapy, immunotherapy methods and treatment with a different compound effective to inhibit one or more of lymphangiogenesis, lymphatic metastasis and angiogenesis (either by way of gene therapy mediated delivery or direct delivery of the compound). From the foregoing, it can be appreciated that the compositions and methods of the present invention offer advantages in providing a means for sustained delivery of a soluble form of VEGFR3 to a subject.

All patent and literature references cited in the present specification are hereby incorporated by reference in their entirety.

Materials and Methods

Immunoprecipitation: Western blots of virus produced sVEGFR3 were carried out using conditioned media, collected 48 hours after infection and heated to 60° C. for 30 min. to inactivate the virus. Serum was collected from mice prior to sacrifice. Both conditioned media and serum were diluted 1:10 with PBS and subjected to standard immunoprecipitation procedures using monoclonal anti-human plasminogen. Proteins were transferred to polyvinylidene difluoride (PVDF) membranes (Immobilon-P™; Millipore, Bedford, Mass.) in transfer buffer (25 mM Tris-HCl pH 7.4, 192 mM glycine and 15% methanol) using a Trans-Blot apparatus (BioRad) for 2 h at 60 V. Protein binding sites on the membranes were blocked by incubating membranes overnight in TNT buffer [10 mM Tris-HCl pH 7.5, 100 mM sodium chloride, 0.1% (v/v) Tween 20 (Sigma)] containing 3% nonfat, powdered milk (blocking buffer). Membranes were incubated with HRP-conjugated goat anti-human plasminogen (Cedarlane Laboratories) at 0.08 mg/ml) for 1 h, washed in TNT buffer with changes every 5 min for 30 min.

Cell lines and Transfections: A human prostate cancer carcinoma cell line, PC-3, and a human melanoma cell line, A375, were purchased from ATCC. PC-3-mlg2 and A375-mln1 are sub-lines of PC-3 and A375 respectively, established by in vivo selection of lymph node metastases from PC-3 or A375 subcutaneous-tumor bearing mice. PC-3-mlg2-VEGFC was a sub-line of PC-3-mlg2, established by transduction with a lentiviral vector encoding human VEGF-C. The above tumor cell lines were maintained in RPMI-1640 medium supplemented with 2 mM 1-glutamine, 100 U/ml penicillin, 100 μg/ml streptomycin, and 10% fetal bovine serum (GIBCO, Grand Island, N.Y.). A human renal clear cell carcinoma cell line, Caki-2, was purchased from ATCC and maintained in McCoy's 5A medium supplemented with 2 mM 1-glutamine, 100 U/ml penicillin, 100 μg/ml streptomycin, and 10% fetal bovine serum (ATCC, Manassas, Va.). The tumor cell lines were transduced with a lentiviral vector expressing the firefly luciferase reporter gene. BaF3NEGFR3-EpoR cells comprise a murine B-cell line stably expressing a chimeric receptor comprised of the extracellular domain of VEGFR-3 and the intracellular domain of erythropoietin receptor. It was maintained in Dulbeco's Modified Essential medium supplemented with 5% fetal bovine serum (GIBCO, Grand Island, N.Y.).

A human lung cancer cell line NCI-H460-LNM35 (i.e., LNM35) was established and maintained as previously described (Kozaki et al., Cancer Res., 60:2535-2540, 2000). LNM35/EGFP and LNM35/Luciferase (LNM35/Luc) cells Were established by infecting LNM35 cells with AAV-EGFP or AAV-Luc viruses, and EGFP+ or Luc+clones were isolated by means of limiting dilution.

Recombinant AAV Vector construction: Standard cloning methods were used for pAAV-CAG-VEGFR-3Fc-WPRE-BGHpA plasmid construction. Initially an rAAV vector plasmid, pAAV-CAG-EGFP-WPRE-BGHpA, that encodes enhanced green fluorescent protein (EGFP) under the control of the CAG promoter was constructed by replacing the liver specific promoter (LSP) of the plasmid pAAV-LSP-EGFP-WPRE-BGHpA with the CAG promoter from the plasmid pBacMam-2 (Novagen, Madison, Wis.). Briefly, the plasmid pAAV-LSP-EGFP-WPRE-BGHpA was cut with Not I, blunted using T4 polymerase, then cut with Xho I, dephosphorylated using Calf Intestinal Phosphatase and a 4902 bp fragment isolated. pBacMam-2 was cut with Xho I, then Hpa I to isolate a 1780 bp fragment. The two fragments were ligated together using a Rapid Ligation Kit (Roche Applied Science: Mannheim, Germany) to generate pAAV-CAG-EGFP-WPRE-BGHpA. To create pAAV-CAG-VEGFR-3Fc-WPRE-BGHpA (SEQ ID NO: 6), the soluble VEGFR-3 receptor Fc fusion encompassing Ig-like domains 1-3 of the human VEGFR-3 receptor fused to human IgG1 heavy chain, was removed from the plasmid pIgPlus-FLT4 (123) Fc using Hind III digestion, restriction sites blunted using T4 polymerase and then ligated into the pAAV-CAG-EGFP-WPRE-BGHpA backbone following removal of the EGFP cDNA with Stu I.

A second recombinant AAV vector (AAV.CAG.VEGFR-3-Ig) was constructed for the experiments with the LNM tumor cell line. The AAV vector psub-CAG-WPRE was cloned by substituting the CMV promoter fragment of psub-CMV-WPRE (Paterna et al., Gene Ther., 7:1304-1311, 2000) with the CMV-chicken beta-actin insert (Niwa et al., Gene, 108:193-199, 1991). The cDNA encoding the soluble VEGFR-3-Ig fusion protein was cloned as a blunt end fragment into the of psub-CAG-WPRE plasmid, and the recombinant AAV viruses (AAV serotype 2) were produced as previously described (Karkkainen, Proc. Natl. Acad. Sci., 98:12677-12682, 2001). HeLa cells were used for expression analysis after transduction with AAV-VEGFR-3-Ig according to standard protocol.

Recombinant AAV vector preparation: Sub-confluent human embryonic kidney 293 cells were co-transfected using the calcium phosphate method with the AAV transfer plasmid and an AAV helper plasmid and pXX-6 (Xiao et al. 1998). For packaging of the AAV vector into the AAV-2 serotype, the AAV helper plasmid pUC-ACG (Xiao et al. 1998) was used and for the AAV-8 serotype the helper p5e18-VD2/8 employed (Gao et al. 2002). Forty-eight hours post-transfection, cells were harvested using PBS/EDTA (10 mM) and lysed by three freeze/thaw cycles. Lysates were treated with benzonase for 15 min at 37° C. and cellular debris was removed by centrifugation. The cleared cell lysate was fractionated by ammonium sulfate precipitation and the rAAV virions were isolated on two sequential CsCl gradients. The gradient fractions containing rAAV were dialyzed against sterile PBS containing CaCl₂ and MgCl₂, and stored at −80° C.

Recombinant Adenoviral Vector Construction and Preparation: The recombinant adenoviruses expressing VEGFR-3-Ig fusion protein (AdVEGFR-3-Ig) or β-galactosidase were constructed as previously decribed (Karpanen et al., Cancer Res., 61:1786-1790, 2001; and International publication no. WO 02/060950). Briefly, the cDNA coding for the VEGFR-3-Ig fusion protein was subcloned into the pAdCMV plasmid, constructed by subcloning the human cytomegalovirus immediate-early promoter, the multiple cloning site, and the bovine growth hormone gene polyadenylation signal from the pcDNA3 (Invitrogen) into the pAdBglII vector. The adenoviruses were produced as described in Laitinen et al., Hum. Gen. Ther., 9:1481-1486, 2001.

Determination of AAV titers: rAAV preps were treated with DNaseI to degrade any unencapsidated DNA and then treated with proteinase K (0.25 mg/ml) in the presence of 0.5% SDS and 10 mM EDTA to liberate the rAAV genomes, followed by phenol chloroform extraction and ethanol precipitation. Viral-DNA was denatured in alkali and applied to a nylon membrane. Dilutions of the corresponding vector plasmid were used as standards to determine the rAAV virion copy number. A radioactive probe specific for the rAAV transgene was hybridized to DNA on the filter and the filter was exposed to film followed by quantification of radioactivity by a β-counter (1450 Micobeta Trilux, PerkinElmer, Inc., Wellesley, Mass.).

sVEGFR3 Bioassay: A bioassay to investigate the blockade of VEGF-C biological activity has been described previously (Makinen et al Nat. Medicine, 2001 7:199-205). Briefly, BaF3NEGFR3-EpoR cells were seeded at 1e4 in 96-well titer plates and incubated overnight in 5% FBS-containing media. The following day, cells were stimulated with 100 ng/ml recombinant human VEGF-C (RnD Systems, Minneapolis, Minn.) in the presence of increasing concentrations of AAV-generated sVEGFR3-Fc containing conditioned media. As a control, cells were stimulated with VEGF-C and conditioned media from vector control-transduced cells. After 72 hrs, VEGF-C-mediated cell proliferation was measured by WST-8 tetrazolium salt using the Cell Counting Kit-8 (Dojindo Laboratories, Kumamato, Japan) according to the manufacturer's recommendations.

sVEGFR3 Detection by Immunoblotting and ELISA: An aliquot of conditioned media harvested from recombinant AAV-sVEGFR3-transduced HuH7 cells, or 100 ng of recombinant human VEGFR3-Fc protein (RnD Systems, Minneapolis, Minn.), were resolved using NuPage Bis-Tris gels and MOPS buffer by 4-12% SDS-PAGE (Invitrogen, Carlsbad, Calif.). Resolved proteins were transferred onto nitrocellulose for 1 hr in 20% methanol-containing transfer buffer (Invitrogen). Membranes were blocked for 1 hr in Tris-buffered saline (TBS) containing 5% BSA and 0.2% Tween-20 (ICN Pharmaceuticals, Inc., Costa Mesa, Calif.), and then probed with 50 ng/ml biotinylated goat anti-VEGFR3 antiserum (RnD Systems, Minneapolis, Minn.) for 1 hr. The blots were washed extensively with TBS-5% BSA, probed with HRP-conjugated-streptavidin (BD Pharmingen) for 1 hr, and subsequently visualized by enhanced chemiluminescence using the Supersignal substrate (Pierce, Rockford Ill.).

For quantification of sVEGFR3 using a sandwich ELISA, 96-well microtiter plates were coated with anti-human IgG capture antibody (Bethyl Laboratories, Montgomery, Tex.) in 0.1M carbonate pH 9.6 buffer and incubated overnight at 4° C. The plates were washed extensively with PBS-0.05% Tween-20; and blocked with PBS-1% BSA-0.05% Tween-20 buffer for 1 hr. Recombinant human VEGFR3-Fc protein (RnD Systems, Minneapolis, Minn.) was used for standard curves after serial dilutions. Samples and the standard were incubated in the wells for 1 hr, washed extensively, and then incubated with 50 ng/ml biotinylated goat anti-human VEGFR3 antiserum (RnD Systems, Minneapolis, Minn.) for 1 hr. After extensive washing, the samples were incubated with HRP-conjugated streptavidin (BD Pharmingen) for 1 hr, washed again, and then detected using Sure Blue TMB substrate (KPL, Gaithersburg, Md.) at 450/650 nm optical density. The detection sensitivity for sVEGFR3-Fc using this ELISA is approximately 1-5 ng/ml.

VEGF-C Detection by ELISA: A solid-phase capture ELISA was used to quantify human VEGF-C protein from serum and tissue culture supernatants. Briefly, 96-well plates were coated with 5 μg/ml recombinant VEGFR3-Fc protein (RnD Systems, Minneapolis, Minn.) in 0.1M carbonate pH 9.6 buffer and incubated overnight at 4° C. The plates were washed extensively with PBS-0.05% Tween-20, and blocked with PBS-1% BSA-0.05% Tween-20 buffer for 2 hr. Recombinant human VEGF-C protein (RnD Systems, Minneapolis, Minn.) was used for standard curves after serial dilutions. VEGF-C-containing samples and the standard were incubated in the wells for 1 hr, washed extensively, and then incubated with 1:500 diluted rabbit anti-human VEGF-C antiserum (obtained from K. Alitalo, Univ. Helsinki, Finland) for 1 hr. After extensive washing, the wells were incubated with 1:1000 diluted HRP-conjugated donkey anti-rabbit IgG antibody (Amersham Biosciences, Piscataway, N.J.) for 1 hr, washed again, and then detected using Sure Blue TMB substrate (KPL, Gaithersburg, Md.) using a microplate reader with an optical density of 450 nm.

RT-PCR detection of VEGF-C: Cultured cells were collected at subconfluency, or resected tumors were flash frozen in liquid nitrogen and stored at −80° C. Frozen cell pellets and pieces of frozen, unthawed tumors were homogenized in TRIzol reagent (Invitrogen Life Technologies, Carlsbad, Calif.) using the PowerGen 125 homogenizer (Fisher Scientific, Hampton, N.H.) to isolate total RNA. Genomic DNA was removed from the samples using DNA-free (Ambion, Austin, Tex.). Samples were stored at −80° C. prior to TaqMan analysis. RNA samples were converted to cDNA and analyzed by quantitative PCR using ABI's TaqMan One-Step RT-PCR Master Mix Reagents Kit (Applied Biosystems, Foster City, Calif., Cat# 4309169) and Assay-on-Demand reagents for hVEGF-C (ABI Cat# Hs00153458_m1), mVEGFC (ABI Cat# Mm00437313 m1), hGAPDH (ABI Cat# Hs99999905_ml), and mGAPDH (ABI Cat# Mm99999915_g1). 400 ng of RNA were included in 50 μl reactions and analyzed in triplicate using recommended conditions and settings on an ABI PRISM 7700 instrument. Relative VEGF-C mRNA expression was analyzed using the delta-delta Ct method (ABI User Bulletin #2, P/N 4303859). Human VEGF-C was normalized with human GAPDH.

Quantitative Detection of Human Tumor Cell Metastasis: The detection of human tumor cells in mouse lymph nodes was based on the quantitative detection of human alu sequences present in mouse lymph nodes DNA extracts (Zijlstra et al., Cancer Res., 62:7083-7092, 2002; Salgaller, M., Curr. Opin. Mol. Ther., 5:657-667,2003. Briefly, genomic DNA was extracted from harvested tissue using the Puregene DNA purification system (Centra Systems, Minneapolis, Minn.). To detect human cells in the mouse tissues, primers specific for human alu sequences were used to amplify the human alu repeats present in genomic DNA that was extracted from the mouse lymph nodes. The real-time PCR used to amplify and detect alu sequences contained 30 ng of genomic DNA, 2 mm MgCl2, 0.4 μM each primer, 200 μM DNTP, 0.4 units of Platinum Taq polymerase (Invitrigen corp, Carlsbad Calif.) and a 1: 100,000 dilution of SYBR green dye) molecular Probes, Eugene, Oreg.). Each PCR was performed in a final volume of 10 ul under 10 ul of mineral oil with the iCycler iQ (Bio-Rad lab, Hercules, Calif.) under the following conditions: polymerase activation at 95° C. for 2 min followed by 30 cycles at 95° C. for 30 s, 63° C. for 30 s, and 72° C. for 30 s. A quantitative measure of amplifiable mouse DNA was obtained through amplification of the mouse GAPDH genomic DNA sequence with mGAPDH primers using the same conditions described for alu. To approximate the actual number of tumor cells present in each tissue sample, a standard curve was generated through quantitative amplification of genomic DNA extracted from a serial dilution of human tumor cells mixed in tissue homogenates. By interpolating the alu signal from experimental samples with standard curve which was generated through quantitative amplification of genomic DNA extracted from a serial dilution of human tumor cells mixed with animal organ homogenates, the actual number of tumor cells/lymph node pool (six lymph nodes from each mouse) determined.

In Vivo Gene Transfer of sVEGFR3: Six- to eight-week-old female NCR nu/nude mice were obtained from Taconic (Germantown, N.Y.). All mice were housed under SPF conditions and treated according to the ILAR Guide for the Care and Use of Laboratory Animals. Mice (n=6) were injected with a single dose of 1e11 virus particles of AAV-sVEGFR3-Fc into the quadricep muscle using a dosing volume of 50 μl/muscle. For liver-directed gene transfer, mice received a single tail-vein injection (200 μl dosing volume) of the indicated amounts of AAV-sVEGFR3-Fc. Mice were bled by alternate retro-orbital puncture on scheduled intervals to measure the serum levels of human sVEGFR3-Fc by ELISA.

Recombinant adenoviruses expressing the VEGFR-3-Ig fusion protein (AdVEGFR-3-Ig) or β-galactosidase (AdLacZ) (1.0e+9 PFU per mouse) were administered via the tail vein one day after the tumor implantation. For the titration experiment, different doses of AdVEGFR-3-Ig (1.0e+9, 1.2e+8, 1.5e+7 or 2.0e+6 PFU) were used. Blood was collected from both the treated and control mice one week after the treatment, and the serum concentration of VEGFR-3-Ig was determined by ELISA. In the ear tumor experiment, recombinant adenoviruses were administered via the tail vein 1 day before the tumor implantation.

Xenotransplantation and metastasis detection: All experiments performed on animals were in accordance with institutional guidelines. For selection of metastatic PC-3 variants, approximately 3×10⁶ luciferase-expressing PC-3 cells in 50 μl of serum-free medium were implanted in the subcutaneous tissue of the dorsal flank of 7-9 week old female NCR nu/nude mice (one tumor per mouse, n=10). Tumors were measured with digital calipers, and the tumor volume (as cubic millimeters) were calculated as follows: volume=length×width² ×0.5. Mice were euthanized after 6 weeks and the internal organs including the axillaries and inguinal lymph nodes from both sides were collected and analyzed by bioluminescence imaging. Briefly, the mice were administered with luciferin substrate (Xenogen Corp., Alameda, Calif.) at a dose of 1.5 mg/g mouse body weight by intraperitoneal injection. Fifteen minutes after substrate injection, the mice were euthanized; the lymph nodes were collected and placed in Petri dish for bioluminescence imaging analysis. Lymph nodes with bioluminescence CCD counts above 1e⁵, detected by bioluminescence imaging analysis (Xenogen), were collected for establishment of primary culture. Briefly, the lymph nodes were minced and incubated with 0.5% trypsin at 37° C. for 15 min. The reaction was stopped by adding 10% FBS-containing medium. The solution was collected and placed in a culture dish. Tumor cells were selected by repeated trypsinization every two days. After 5 passages, the tumor cells were harvested. Approximately 3×10⁶ cells in 50 μl of serum-free medium were implanted in the subcutaneous tissue of the dorsal flank of female NCR nu/nude mice for outgrowth and further metastatic selection. PC-3-mlg2 tumor cells were established after two rounds of in vivo selection as described above. A375-mln2 tumor cells were selected following one round of selection using similar procedures as described above. Samples of tumors were snap-frozen in liquid nitrogen and stored at −70° C. for RT-PCR and protein analysis, or fixed immediately in 4% paraformaldehyde for further histological analysis.

Tumor implantation and treatment with either AdVEGFR-3-Ig or AdLacZ were performed as previously described (He et al., J. Natl Cancer Inst., 94:819-825, 2002). Briefly, tumors were excised one, two or three weeks after tumor implantation. Mice were allowed to recover, and sacrificed within seven weeks after the removal of primary tumors. Tissues were collected and processed for histology. Lymph nodes were measured and also weighed. In separate experiments, LNM35/EGFP cells (1-5×105 in 30 μl) were injected subcutaneously into the ears of the nude mice, and mice were treated as above (n=6 for each group). Tumor-transplanted ears were analyzed within two weeks.

In the LNM35 tumor experiments using the AAV, LNM35 tumors were implanted in nude mice (Balb/c) or SCID mice three weeks after the first administration of AAV-VEGFR-3-Ig. Mice were sacrificed within 5 weeks, and tumors, some internal organs including the lungs, and axillary lymph nodes were collected and analysed under a dissecting LEICA MZFLIII microscope for EGFP signal. The lymph node volumes were calculated as previously described (Warri et al., J. Intl Cancer Inst, 85:1412-1418, 1993). Samples were processed as described above for further histology. In mice with BrdU labeling, each mouse was injected intraperitoneally with 0.5 ml BrdU (5 mg/ml Sigma) to mark proliferating cells one hour before sacrifice. Tissues were collected and processed as described above.

Evaluation of lymph node metastasis. In all AAV efficacy studies, mice received AAV recombinant vectors ten days prior to tumor challenge. The animals were bled by alternate retro-orbital puncture on scheduled intervals though out the study to measure the serum levels (+/−sem) of human sVEGFR3-Fc by ELISA. For PC-3 and A375 tumor models, animals were euthanized either five or three weeks post-tumor cell inoculation. For evaluation of lymphogenous metastasis, lymph nodes (including axillaries and inguinal nodes from both sides) were collected from each animal analyzed by bioluminescence imaging as described above. A set of six lymph nodes collected from a naïve mouse was used as negative control in each study. The metastases of each mouse were calculated based on total bioluminescence (CCD counts).

In a separate study, 5×10⁶ Caki-2 tumor cells were administered ten days following AAV vector injection. The lymph nodes (axillaries and inguinal nodes from both sides) were collected from each animal and the length and the width of lymph nodes were measured. The volumes (as cubic milliliters) were calculated as volume=(π/6)×(length×width).

B16 B1/6 Mouse melanoma metastasis model: Female C57B1/6 mice were obtained from Taconic and mice were at 6-8 weeks old at the start of each experiment. Mice were injected with 5×10e4 B16B1/6 cells on day 0 via tail vein with a 27-gauge needle. After 14-21 days, mice were sacrificed and their tumor burden assessed by harvesting the mice lungs and counting the surface tumor metastasis and measuring the weight of the lung. All experiments had 6-10 animals per group. Statistical significance was evaluated using the Student's t-test.

Xenogen Imaging of Tumor Models: In vivo luminescence of tumor bearing mice were monitored by biweekly monitoring of B16F10-luciferase (Xenogen Inc.) injected mice. In brief, Balb/c nu/nu mice were injected with 5×10⁴ or 2×10⁵ cells of B16F10-luc cells via tail vein on day 0. Mice were monitored for tumor burden when necessary by intra-peritoneal injection of excess luciferin substrate at 1.5 mg/g mice weight. Twenty minutes after substrate injection, mice are anesthesized and monitored for in vivo luminescence with Xenogen IVIS Imaging System (Xenogen Inc.) luminescence sensitive CCD camera by dorsal or ventral position. Data is collected and analyzed by Living Image 2.11 software. CCD photon counts are analyzed by Living Image 2.11 and an Excel spreadsheet.

Immunofluorescence Staining: For whole mount staining, tissues were fixed and stained as previously described (He et al., Cancer Res., 64:3737-3740, 2004). Samples were then mounted with Vectashield (Vector Laboratories) and analysed with a Zeiss LSM510 confocal microscope. For staining of tissue sections, paraffin sections (6 μm) of fixed tissue were immunostained with monoclonal antibodies against PECAM-1 (PharMingen) and LYVE-1 as described previously (Karkkainen et al., Nat. Immunol., 5:74-80, 2004; Prevo et al., J. Biol. Chem., 276:19420-19430, 2601). The extracellular domain of human LYVE-1 (residues 1-232, Uniprot Q9Y5Y7) was fused to the Fc part of human IgG1 and produced using the Bac-to-Bac system (Invitrogen). Immunization was started with 0.4 mg protein per rabbit in Freund's complete adjuvant. Booster injections containing Freund's incomplete adjuvant and 0.2 mg protein per rabbit were given after 3, 6, 9 and 12 weeks, followed by terminal bleeding one week after the last booster injection. In some experiments, proliferating cells in the sections were first stained by using the PCNA or BrdU staining kit (Zymed) and then stained for LYVE-1.

Fluorescent microlymphography: The functional lymphatic network surrounding the tumors subcutaneously implanted in the ears was visualized by fluorescent microlymphography using dextran conjugated with fluorescein isothiocyanate (molecular weight: 2000 kDa, Sigma), that was injected intradermally into the ears. The lymphatic vessels were then examined using a dissection microscope.

The following examples illustrate but are not intended in any way to limit the invention:

### EXAMPLE 1

### Construction of a Recombinant AAV Vector Encoding Soluble VEGFR3

In order to evaluate the biological activity (e.g., inhibition of lymphangiogenesis, lymphatic metastasis and angiogenesis) of a human soluble VEGFR3 receptor using gene therapy, recombinant AAV vectors that encode a secreted form of human VEGF-Receptor-3 (sVEGFR3) were constructed. The recombinant AAV vector encoding soluble VEGFR3 set forth in FIG. 2 was constructed by standard techniques described above. The vector, which expresses a soluble form of VEGFR-3, sVEGFR-3Fc, encompasses a sequence encoding the 1-3 Ig-like domains of human VEGFR-3 gene fused in-frame to the human IgG1 heavy chain (Fc) and was cloned downstream of the constitutive CAG promoter and upstream of the Woodchuck Hepatitis Virus Post-transcriptional regulatory element (WPRE) and bovine growth hormone polyadenylation sequence (bGHpA) in an AAV vector.

### EXAMPLE 2

Expression of Soluble Human VEGFR3-Fc Following AAV-Mediated Gene Transfer In Vitro and In Vivo

A. Western Blot Analysis

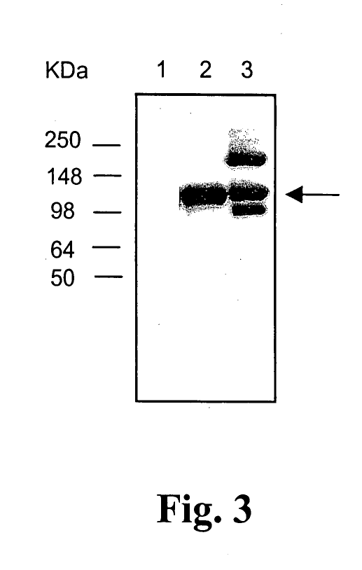

To verify the expression of sVEGFR3-Fc from the AAV vectors, HuH7 hepatocarcinoma cells were transduced with recombinant AAV vectors encoding human sVEGFR3-Fc, or the corresponding AAV control vector, at a multiplicity of infection of 1 e⁵ vector particles per cell. After 48 hr, conditioned media collected from AAV-Null and AAV-sVEGFR3-Fc transduced HuH7 cells or 100 ng of recombinant human VEGFR3-Fc protein were resolved by SDS-PAGE and probed with an anti-VEGFR3 anti-sera. An immunoreactive band with a molecular weight of approximately 115 kDa was detected in the supernatant of AAV-sVEGFR-Fc-transduced cells, but not in AAV-control transduced cells. The band was identical to that detected with recombinant VEGFR3-Fc protein comprised of the entire extracellular domain of human VEGFR-3 (FIG. 3).

B. In Vitro Activity of sVEGFR3 Expressed Using an AAV Vector

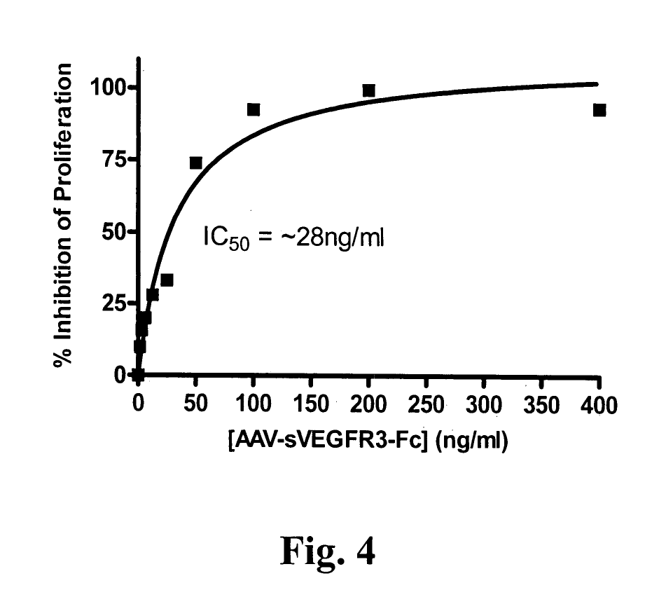

The biological activity of AAV-expressed sVEGFR3 was evaluated in vitro based on VEGFC-induced proliferation in responsive Ba/F3 cells that express a chimeric VEGFR-3/erythropoietin receptor (Makinen et al, 2001 Nat. Med). Dose-dependent inhibition of VEGFC-mediated cell proliferation by soluble human VEGFR3-Fc generated from AAV vectors was evaluated in vitro using BaF3/VEGFR3-EpoR cells, a murine B-cell line stably expressing a chimeric receptor comprised of the extracellular domain of human VEGFR-3 and the intracellular domain of erythropoietin receptor, were seeded at 1 e4 per well in 96-well plates and stimulated with 100 ng/ml recombinant human VEGF-C in the presence of increasing concentrations of conditioned media containing AAV-generated sVEGFR-3. Conditioned media from cells transduced with AAV-control vector were used as control. After 72 hrs, VEGF-C-mediated cell proliferation was measured using a WST-8 tetrazolium salt-based proliferation assay (FIG. 4). The 50% inhibitory concentration (IC⁵⁰) of sVEGFR3-Fc was calculated to be approximately 30 ng/ml in this assay when the cells were stimulated with 100 ng/ml of human VEGF-C for 72 hr.

C. In Vivo Evaluation of sVEGFR3 Expression Using an AAV Vector

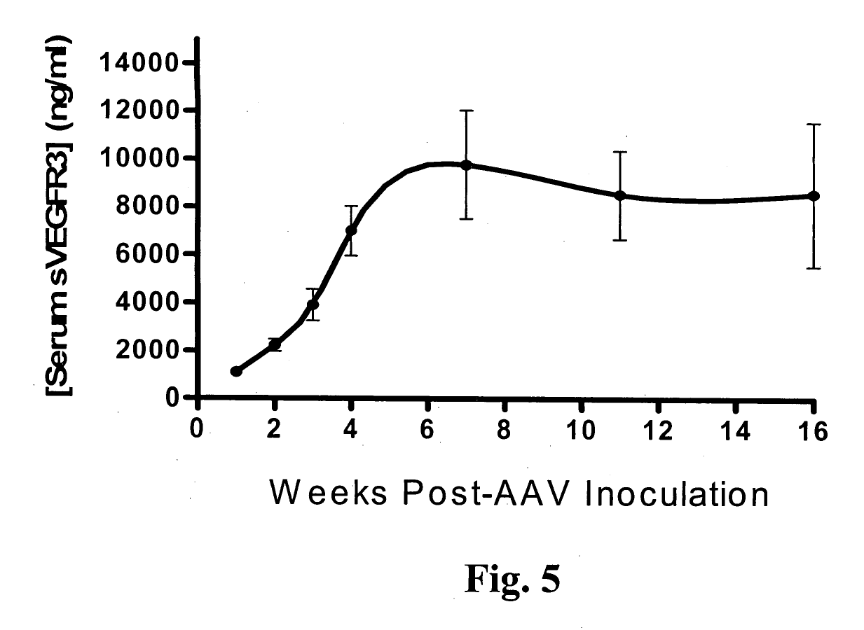

The vivo expression of AAV-expressed sVEGFR3 was analyzed using muscle-directed AAV-mediated gene transfer in immunodeficient mice. A single administration of 1e11 virus particles of AAV-sVEGFR3-Fc into the quadricep muscle of NCR-nu/nu mice resulted in sVEGFR3-Fc serum levels of approximately 1 μg/ml one week following gene transfer (FIG. 5). Serum levels of sVEGFR3-Fc increased over time until steady-state levels of 8-10 μg/ml were reached approximately 4-5 weeks following vector administration with sustained expression of sVEGFR3-Fc detected in the sera of mice for more than 4 months following a single injection of AAV-sVEGFR3-Fc (FIG. 5).

### EXAMPLE 3

In Vivo Evaluation of the Biological Activity of sVEGFR3 Expressed Using an AAV Vector.

In order to investigate the effects of AAV-sVEGFR3-Fc mediated gene transfer in vivo on tumor-induced lymphangiogenesis and lymphogenous metastasis, several metastatic human tumor models were developed, including the PC-3 mlg2 and A375-mln1 models. In these tumor models, subcutaneously inoculated tumor cells preferentially disseminate to the lymph nodes and form metastases. Both PC-3-mlg2 and A375-mln1 tumor variants were established from lymph node metastases of mice bearing subcutaneous tumors from their respective PC-3 and A375 parental cell lines. PC-3-mlg2 was established after two rounds of in vivo selection, whereas A375-mln1 was established after one round of selection, as described above.

A. PC3 Tumor Model

The metastatic tumor cell line, PC-3-mlg2, was established from lymph node metastases of mice bearing subcutaneous PC-3 tumors after two rounds of in vivo selection. The incidences of tumor cells that metastasize to the lymph nodes was 7- to 8-fold greater for PC-3-mlg2 relative to PC-3 cells. The level of mRNA and protein for VEGF-C was doubled in the selected cell line, as shown in Table 2.

TABLE 2
Enhanced lymph node metastasis is associated with increased expression
of VEGFC

| | | VEGFC expression | |
|---|---|---|---|
| | Incidence of lymph node metastases | (Fold increase) | |
| Tumor type | in tumor-bearing mice | mRNA | Protein |
| PC-3 | 10% | 1 | 1 |
| PC-3-mlg2 | 75% | 2 | 2.5 |

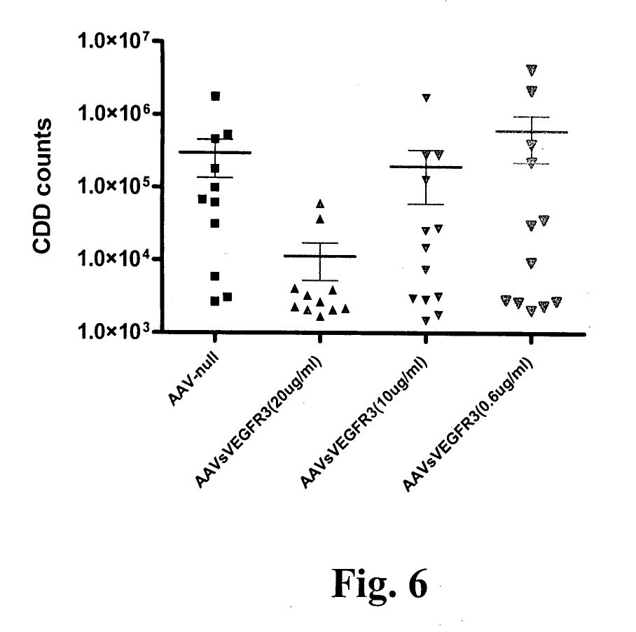

The effect of AAV-expressed sVEGFR3 on lymph node metastases in female NCR nu/nu mice injected intramuscularly with AAV-sVEGFR3-Fc, at 1.5e11, 7e10 or 3e10 of virus particles was evaluated. Control mice were injected with 1.5e11 virus particles of AAV-null. Ten days after AAV administration, mice were challenged subcutaneously with 3e6 PC-3-mlg2 tumor cells that stably express a luciferase reporter gene. Five weeks post-tumor cell challenge, the lymph nodes were collected for evaluation of lymphogenous metastasis using bioluminescence imaging (axillary and inguinal nodes from both sides). The number of luciferase positive (metastatic positive) lymph nodes in each animal were compared among the groups treated with increasing doses of AAV-sVEGFR3 and to the control vector group. High sVEGFR3-Fc serum levels (20 μg/ml) significantly inhibited tumor metastasis to the lymph nodes in the PC-3-mlg2 tumor model. See FIG. 6. As shown in Table 3, two out of eleven mice (18%) developed lymph node metastases following treatment with the highest dose of AAV-sVEGFR3-Fc (1.5e11 vector particles) and eight out of eleven mice (73%) developed lymph node metastases when treated with an equivalent dose of AAV-null vector. The inhibition of metastasis by sVEGFR3-Fc was diminished when the treatment dose of AAV-sVEGFR3-Fc was decreased. The results (shown in Table 3 and FIG. 6) indicate that systemic expression of a soluble VEGFC/D inhibitor following AAV-mediated gene transfer inhibits the spread of metastatic PC-3-mlg2 tumor cells to the lymph nodes in a dose-dependent manner.

TABLE 3
Effect of Systemic expression of AAV-sVEGFR3-Fc on tumor metastasis
to the lymph nodes in immune-deficient mice.

| | | Inhibition |
|---|---|---|
| | | of lymph |
| | No. of animals with lymph | node |
| Groups | node metastasis/total mice | metastasis |
| AAV-null | 8/11 | — |
| AAV-sVEGFR3-Fc, 20 μg/ml | 2/11 | 82% |
| | | (P = 0.01) |
| AAV-sVEGFR3-Fc, 10 μg/ml | 7/13 | 46% |
| | | (P > 0.05) |
| AAV-sVEGFR3-Fc, 0.6 μg/ml | 7/12 | 42% |
| | | (P > 0.05) |

B. A375 Tumor Model

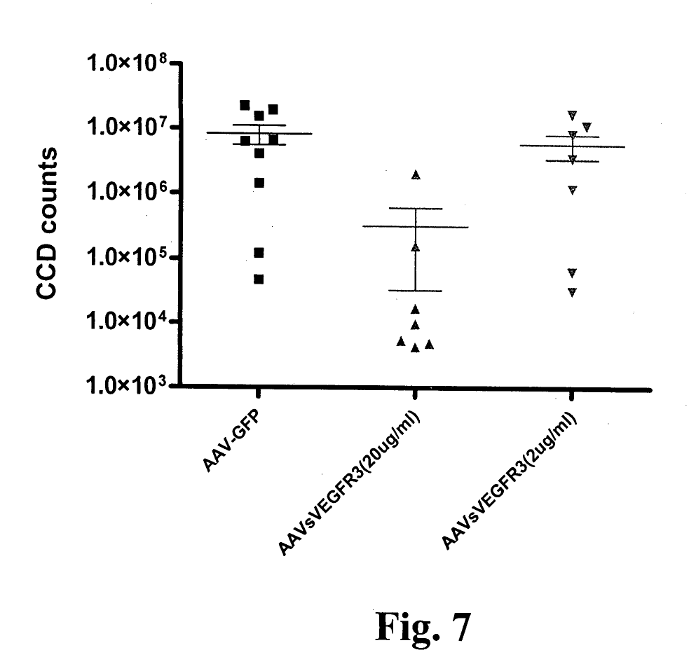

The metastatic tumor cell line, A375-mln1, was established from lymph node metastases of mice bearing subcutaneous A375 tumors after two rounds of in vivo selection. The incidences of tumor cells that metastasize to the lymph nodes was 3-fold greater for A375-mln1 relative to A375 cells. The dose-dependent effect of AAV-sVEGFR3-Fc on inhibition of A375 tumor metastasis to the lymph nodes was evaluated. Female NCR nu/nu mice were injected with 3e11 or 1.5e11 vector particles of AAV-sVEGFR3-Fc. Control mice were injected with 3e11 virus particles of AAV-null vector. Ten days following AAV administration, mice were challenged subcutaneously with 3e6 A375-mln1 tumor cells that stably express a luciferase reporter gene. Three weeks post-tumor cell challenge, the lymph nodes were collected for evaluation of lymphogenous metastasis of using bioluminescence imaging (axillary and inguinal nodes from both sides). The numbers of luciferase positive (metastatic positive) lymph nodes in each animal were compared among the groups treated with AAV-sVEGFR3 and the control vector. High levels (20 μg/ml) of serum sVEGFR3-Fc following gene transfer significantly inhibited tumor metastasis to the lymph nodes in the A375 tumor model (p=0.01) (See FIG. 7.) As described above for the PC3-mlg2 model, systemic expression of sVEGFR3-Fc following AAV-mediated gene transfer inhibited the metastasis of human A375 melanoma tumors to the lymph nodes in a dose-dependent manner (FIG. 7).

C. RCC/Caki-2 Tumor Model

The dose-dependent effect of AAV-sVEGFR3-Fc on inhibition of Caki-2 tumor metastasis to the lymph nodes was evaluated.

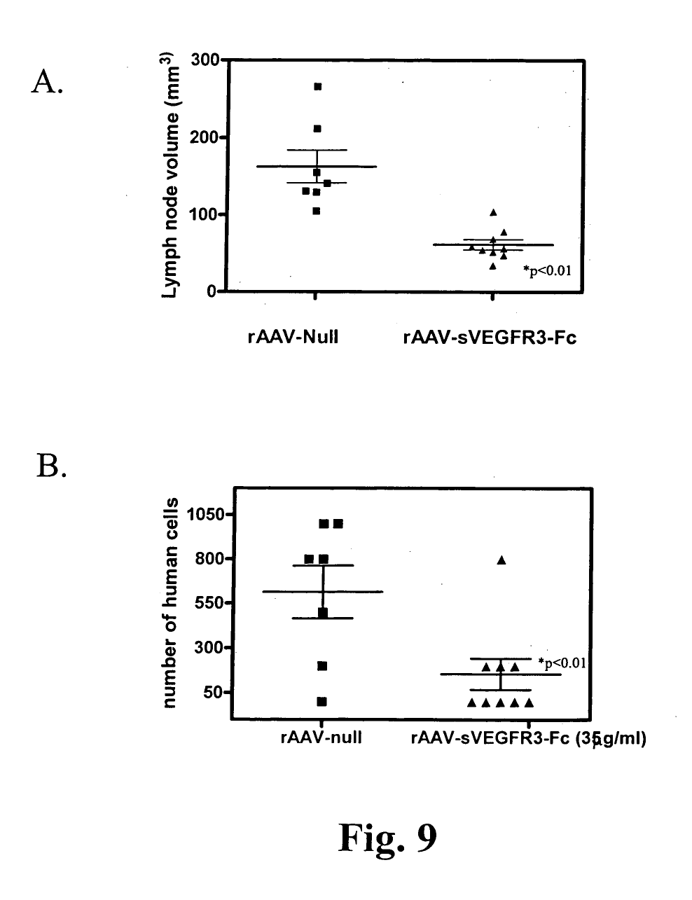

A single injection of rAAV-sVEGFR3-Fc (at either 3×10¹¹ or 1.5×10¹¹ vg/animal) was administered into the muscle of mice ten days prior to Caki-2 metastatic tumor cell challenge. Sustained sVEGFR3-Fc serum levels of approximately 35 μg/ml and 10 μg/ml were detected following rAAV-mediated gene transfer. Two weeks post tumor challenge, lymph nodes (axillaries and inguinal from both sides) were collected for evaluation of lymphogenous metastasis by injecting Lymphazurin (1% isosulfan blue) into the tumor, a dye that specifically stains lymphatic vessels and lymph nodes. Recombinant AAV-sVEGFR3-Fc mediated gene transfer resulted in an approximately 60% (p<0.01) reduction of Caki-2 induced lymph node enlargement versus the control treatment (FIG. 9A). Furthermore, a strong enlargement and increase in number of the draining lymphatic vessels from subcutaneously grown tumors to the draining lymph nodes was observed when Lymphazurin (1% Isosulfan blue) was injected intra-/peri-tumorally into animals treated with the rAAV-Null vector. In contrast, no tumor associated lymphatic vessels were found by Lymphazurin injection in rAAV-sVEGFR3-Fc treated animals. The lack of lymphatic vessels in these rAAV-sVEGFR3-Fc treated animals correlated with a lack of dye-positive lymph nodes.

PCR analysis was performed on lymph nodes isolated from these animals and revealed the presence of more human genome (alu) sequences in control animals than in rAAV-sVEGFR3-Fc treated animals (FIG. 9B). The number of human tumor cells in the lymph nodes calculated from the PCR data was significantly lower in the rAAV-VEGFR3-Fc treated animals than in rAAV-Null injected animals.

A reduction in the number of lymphatic vessels and a 70% inhibition of lymph node metastases was observed in Caki-2-tumor bearing animals that continuously expressed approximately 35 μg/ml of sVEGFR3-Fc in the serum following rAAV-sVEGFR3-Fc gene transfer. Furthermore, lower sVEGFR3-Fc serum levels of approximately 10 μg/ml resulted in reduced inhibition of lymph node metastases in tumor-bearing mice.

### EXAMPLE 4

In Vivo Evaluation of the Effect of Blocking the Biological Activity of AAV-Expressed sVEGFR3 Expressed Using VEGFC

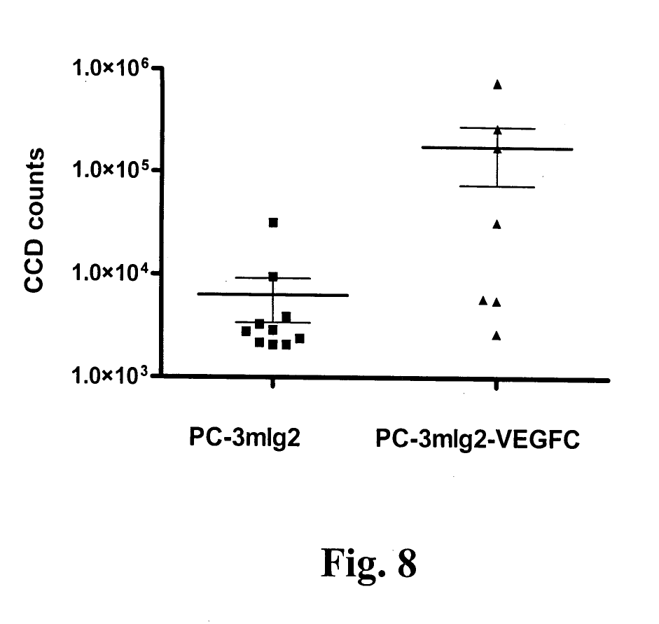

The concentration of AAV-sVEGFR3-Fc required for blocking tumor metastasis to the lymph nodes is dependent on the amount of its ligand, VEGF-C and also VEGF-D, expressed by the primary tumor. In order to verify the mechanism of action of AAV-expressed sVEGFR3-Fc, another tumor model was established using PC-3-mlg2 tumor cells (described in Example 3A) transduced with a lentiviral vector expressing human VEGF-C to generate a cell line expressing increased levels of VEGFC both in vitro and in vivo (designated as PC-3-mlg2-VEGFC; see Table 3). When PC-3-mlg2-VEGFC cells are implanted subcutaneously into nude mice, they demonstrate similar growth kinetics as the parental PC-3-mlg2 cells, however, they express greater than 10-fold more VEGF-C (Table 3). Furthermore, lymph node metastases were rapidly observed in 100% of these animals (data not shown). Female NCR nu/nu mice were injected intramuscularly with 1.5e11 virus particles of AAV-sVEGFR3-Fc. Ten days after AAV administration; mice were challenged subcutaneously with 3e6 of PC-3-mlg2 or PC-3-mlg2-VEGFC tumor cells, respectively. Mice were bled weekly by alternate retro-orbital puncture throughout the study to determine sVEGFR3-Fc serum levels by ELISA (data not shown). Five weeks post-tumor cell inoculation, the lymph nodes were collected for evaluation of lymphogenous metastasis. At a sVEGFR3-Fc serum concentration of 15 μg/ml, 50% more lymph node metastases were found in PC-3-mlg2-VEGFC tumor-bearing animals than in PC-3-mlg2 tumor-bearing animals (p=0.03). See FIG. 8 and Table 4.

TABLE 4

| | Fold increase in VEGF-C | | Fold increase in VEGF-C | |
|---|---|---|---|---|
| | expression (cells) | | expression (tumors) | |
| Tumor type | mRNA | Protein | mRNA | Protein |
| PC-3-mlg2 | 1 | 1 | 1 | 1 |
| PC-3-mlg2-VEGFC | 7.5 | 8 | 16 | 12 |

In summary, the inhibition of lymph node metastasis was nearly 50% (p=0.03) more efficient in PC-3-mlg2 as compared to PC-3-mlg2-VEGFC tumor-bearing animals (FIG. 9) although equal serum levels of sVEGFR-3 were detected in animals in both treatment groups.

### EXAMPLE 5

In Vivo Evaluation of the Biological Activity of sVEGFR3 Expressed Using an AV Vector.

Fluorescently labelled LNM35 tumor cells were implanted in the subcutaneous tissue of mouse ear, and analyzed after various time points. Whole mount staining for LYVE-1 revealed extensive lymphatic vessel sprouting towards the EGFP expressing tumor cells. Lymphatic vessels often grew around single tumor cells or tumor cell masses, enveloping the tumor cells. Similar elongated lymphatic endothelial cells were observed around clusters of tumor cells when these cells were co-cultured in vitro. In mice treated with AdVEGFR-3-Ig, the lymphatic sprouting was inhibited, but the tumor cells could still co-opt pre-existing lymphatic vessels. The treatment inhibited tumor-associated lymphatic vessel growth and dye leakage from the newly formed vessels as seen in microlymphangiography of the subcutaneously implanted tumor areas.

The spread of tumor cells via the lymphatic vessels was evaluated. Mice bearing subcutaneous LNM35/EGFP tumors were anesthetized, a skin flap containing the collecting lymphatic vessels draining the tumor was inverted and analyzed by fluorescence microscopy at week 5 after tumor implantation. The draining lymphatic vessels were clearly dilated. For comparison, the collecting lymphatic vessels in the skin of a mouse without tumor were visualized using FITC-dextran microlymphography. Both single tumor cells and tumor cell masses were observed in the lymphatic vessels of the untreated mice, but not in the AdVEGFR-3-Ig treated tumor-bearing mice.

To assess whether the dilation of the collecting lymphatic vessels was accompanied by lymphatic endothelial cell proliferation, a bolus of BrdU was administered 1 hour before the mice were sacrificed. About 8% of the lymphatic endothelial cells were proliferating in the peri-tumoral draining lymphatic vessels of the control mice ase seen by double-labeling for LYVE-1 and nuclear BrdU. However, proliferating lymphatic endothelial cells were only rarely observed in the AdVEGFR-3-Ig treated mice.

### EXAMPLE 6

Dose-dependent Inhibition of Macrometastasis by AdVEGFR-3-Ig and AAV-VEGFR-3-Ig

The effect of different levels of circulating VEGFR-3-Ig on lymphatic metastasis was investigated. Luciferase-expressing LNM35 tumor-bearing SCID mice were injected with different doses of AdVEGFR-3-Ig via the tail vein. Serum concentrations of the VEGFR-3-Ig fusion protein determined one week after injection correlated with the adenovirus dose. Bioluminescent signals emitted from the lymph nodes of the treated and control mice were quantified in photons/sec/cm2/sr five weeks after tumor implantation. Some suppression of lymph node metastasis was obtained even with the lowest dose of 2.0e+6 PFU of AdVEGFR-3-Ig.

Dramatic increase of lymph node size (“macrometastasis”) occurred in all mice in the control group ( 6/6), and also in some of the mice receiving AdVEGFR-3-Ig (⅙ mice with 1.5e+7 PFU; 2/6 mice with 2.0e+6 PFU). Although macrometastasis was rare in tumor-bearing mice receiving high doses of AdVEGFR-3-Ig (1.0e+9 PFU), micrometastasis as determined by the presence of the luciferase signal in the lymph nodes still occurred in most of the treated mice.

When the tumor was removed 2 weeks after implantation, only ⅕ of the control SCID mice developed macrometastasis by week 6. However, when the tumor was removed 3 weeks after implantation, ⅚ of the control mice developed macrometastasis. No macrometastases were observed in the AdVEGFR-3-Ig (1.0e+9 PFU) treated mice. Even more importantly, no micrometastases were observed in any of the AdVEGFR-3-Ig treated mice when the tumor was removed at week 2 or 3.

Inhibition of lymphatic metastasis was also achieved in LNM35 tumor-bearing mice treated with the AAV-VEGFR-3-Ig. In nude mice receiving AAV.CAG.VEGFR-3-Ig (1.0e+11 vp) by the intramuscular delivery, the serum concentration of VEGFR-3-Ig at week 3 was 393.4±185.2 ng/ml (n=10). There was only a slight decrease of the circulating VEGFR-3-Ig nine weeks after the administration of the recombinant AAV viruses (333.4±151.2 ng/ml, n=9).

SCID mice that were injected i.m. with AAV-VEGFR-3-Ig (4.0e+11 vg) had 2.02±0.58 μg/ml (n=12) VEGFR-3-Ig in the circulation three weeks after virus adminstration. There was a significant difference in lymph node volume between the treated and control mice five weeks after tumor inoculation. In the AAV-VEGFR-3-Ig group, the lymph node volume was 2.51±1.61 mm³ (n=12), whereas it was 13.10±14.59 mm³ in the control group (n=6, P=0.0209, unpaired t test). In the mice treated with AAV-VEGFR-3-Ig, macroscopically evident metastasis, which was present in the control group receiving AAV-EGFP ( 2/6, the rest contained micrometastasis), was not observed.

### EXAMPLE 7

Tumor Lymphangiogenesis Occurs Later than Angioienesis.

The tumors were excised at one, two or three weeks after xenotransplantation into nude mice in order to determine when lymphangiogenesis occurs during tumor growth. The lymphatic vessels in the tumors were analyzed by immunostaining using antibodies against the lymphatic endothelial marker LYVE-1. No lymphatic vessels were seen in the tumors or peritumoral areas at week 1, while some were detected in week 2 tumors. However, there was a dramatic increase of lymphatic vessels in week 3 tumors and peri-tumoral tissues. The average number of intra-tumoral LYVE-1 positive vessels was determined (week 2 tumor: 2.07±3.22 vessels/grid, n=6; week 3 tumor: 12.17±2.63; mean±SD, n=6). There was a significant increase in the lymphatic vessel density between week 2 and 3 as determined by the unpaired T test (the two tailed P value=0.0001). Intratumoral lymphatic vessels were not observed in tumors from AdVEGFR-3-Ig treated mice at week 1 or 2, and only few were detected at week 3 (0.37±0.64, mean±SD, n=10).

Staining of tumor sections for PECAM-1, a panendothelial marker, showed robust angiogenesis at all stages of tumor growth, and in both intra- and peri-tumoral tissues. The intra-tumoral vessel density, as determined from three microscopic fields of the highest vessel density, was 43.08±7.41 vessels/grid (n=4), 30.14±7.05 (n=5), and 31.04±5.05 (n=5) in week 1, 2 and 3 tumors from the control mice, and 39.28±10.53 vessels/grid (n=4), 28.40±6.53 (n=6) and 26.63±7.05 (n=6), respectively, in mice treated with AdVEGFR-3-Ig. Thus there was no significant difference in the blood vessel density between tumors from the AdVEGFR-3-Ig treated and control mice at these timepoints.

The spread of tumor cells to regional lymph nodes was also evaluated. No lymph node metastasis was detected in the control group when the tumors were excised at week 1. Lymph node metastasis was also rarely detected when tumors were excised at week 2 ( 1/13). However, about two thirds of the mice developed lymph node metastasis after tumors were removed at week 3 ( 8/13). Therefore, similar to the SCID mice, tumor cell spread to regional lymph nodes was initiated primarily between weeks 2 and 3 after the xenotransplantation into nude mice. No lymph node metastasis was detected in the AdVEGFR-3-Ig treated mice by histological analysis. Interestingly, tumor cells were able to proliferate in lymphatic vessels and they established metastatic foci in the draining lymphatic vessels of both SCID mice and nude mice.

TABLE 5
Brief Table of the Sequences.

| | |
|---|---|
| SEQ | DESCRIPTION |
| ID NO | |

| | |
|---|---|
| 1 | coding sequence for a biologically active form of sVEGFR3: |
| | 1719 b.p. in length |
| | Sequence 1-987 VEGFR-3 (Flt-4) receptor Ig-like domains 1-3 |
| | Sequence 988-1022 Linker region |
| | Sequence 1023-1719 IgG1 Fc |
| | ATGCAGCGGGGCGCCGCGCTGTGCCTGCGACTGTGGCTCTGCCTGGGACTCCTGGACGGCCTGGTGAGT |
| | GGCTACTCCATGACCCCCCCGACCTTGAACATCACGGAGGAGTCACACGTCATCGACACCGGTGACAGC |
| | CTGTCCATCTCCTGCAGGGGACAGCACCCCCTCGAGTGGGCTTGGCCAGGAGCTCAGGAGGCGCCAGCC |
| | ACCGGAGACAAGGACAGCGAGGACACGGGGGTGGTGCGAGACTGCGAGGGCACAGACGCCAGGCCCTAC |
| | TGCAAGGTGTTGCTGCTGCACGAGGTACATGCCAACGACACAGGCAGCTACGTCTGCTACTACAAGTAC |
| | ATCAAGGCACGCATCGAGGGCACCACGGCCGCCAGCTCCTACGTGTTCGTGAGAGACTTTGAGCAGCCA |
| | TTCATCAACAAGCCTGACACGCTCTTGGTCAACAGGAAGGACGCCATGTGGGTGCCCTGTCTGGTGTCC |
| | ATCCCCGGCCTCAATGTCACGCTGCGCTCGCAAAGCTCGGTGCTGTGGCCAGACGGGCAGGAGGTGGTG |
| | TGGGATGACCGGCGGGGCATGCTCGTGTCCACGCCACTGCTGCACGATGCCCTGTACCTGCAGTGCGAG |
| | ACCACCTGGGGAGACCAGGACTTCCTTTCCAACCCCTTCCTGGTGCACATCACAGGCAACGAGCTCTAT |
| | GACATCCAGCTGTTGCCCAGGAAGTCGCTGGAGCTGCTGGTAGGGGAGAAGCTGGTCCTGAACTGCACC |
| | GTGTGGGCTGAGTTTAACTCAGGTGTCACCTTTGACTGGGACTACCCAGGGAAGCAGGCAGAGCGGGGT |
| | AAGTGGGTGCCCGAGCGACGCTCCCAGCAGACCCACACAGAACTCTCCAGCATCCTGACCATCCACAAC |
| | GTCAGCCAGCACGACCTGGGCTCGTATGTGTGCAAGGCCAACAACGGCATCCAGCGATTTCGGGAGAGC |
| | ACCGAGGTCATTGTGCATGAGGATCCCATCGAAGGTCGTGGTGGTGGTGGTGGTGATCCCAAATCTTGT |
| | GACAAACCTCACACATGCCCACTGTGCCCAGCACCTGAACTCCTGGGGGGACCGTCAGTCTTCCTCTTC |
| | CCCCCAAAACCCAAGGACACCCTCATGATCTCCCGGACCCCTGAGGTCACATGCGTGGTGGTGGACGTG |
| | AGCCACGAAGACCCTGAGGTCAAGTTCAACTGGTACGTGGACGGCGTGGAGGTGCATAATGCCAAGACA |
| | AAGCCGCGGGAGGAGCAGTACAACAGCACGTACCGTGTGGTCAGCGTCCTCACCGTCCTGCACCAGGAC |
| | TGGCTGAATGGCAAGGAGTACAAGTGCAAGGTCTCCAACAAAGCCCTCCCAGCCCCCATCGAGAAAACC |
| | ATCTCCAAAGCCAAAGGGCAGCCCCGAGAACCACAGGTGTACACCCTGCCCCCATCCCGGGATGAGCTG |
| | ACCAAGAACCAGGTCAGCCTGACCTGCCTAGTCAAAGGCTTCTATCCCAGCGACATCGCCGTGGAGTGG |
| | GAGAGCAATGGGCAGCCGGAGAACAACTACAAGGCCACGCCTCCCGTGCTGGACTCCGACGGCTCCTTC |
| | TTCCTCTACAGCAAGCTCACCGTGGACAAGAGCAGGTGGCAGCAGGGGAACGTCTTCTCATGCTCCGTG |
| | ATGCATGAGGCTCTGCACAACCACTACACGCAGAAGAGCCTCTCCCTGTCTCCGGGTAAATGA |
| 2 | Corresponding amino acid sequence to the coding sequence for a |
| | biologically active form of sVEGFR3 presented as SEQ ID NO:1: |
| | 572 aa in length |
| | 1-328 aa VEGFR-3 (Flt-4) receptor Ig-like domains 1-3 |
| | 329-341 aa Linker |
| | 342-572 aaIgG1 Fc |
| | MQRGAALCLRLWLCLGLLDGLVSGYSMTPPTLNITEESHVIDTGDSLSISCRGQHPLEWAWPGAQEAPA |
| | TGDKDSEDTGVVRDCEGTDARPYCKVLLLHEVHANDTGSYVCYYKYIKARIEGTTAASSYVFVRDFEQP |
| | FINKPDTLLVNRKDAMWVPCLVSIPGLNVTLRSQSSVLWPDGQEVVWDDRRGMLVSTPLLHDALYLQCE |
| | TTWGDQDFLSNPFLVHITGNELYDIQLLPRKSLELLVGEKLVLNCTVWAEFNSGVTFDWDYPGKQAERG |
| | KWVPERRSQQTHTELSSILTIHNVSQHDLGSYVCKANNGIQRFRESTEVIVHEDPIEGRGGGGGDPKSC |
| | DKPHTCPLCPAPELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQVYTLPPSRDEL |
| | TKNQVSLTCLVKGFYPSDIAVEWESNGQPENNYKATPPVLDSDGSFFLYSKLTVDKSRWQQGNVFSCSV |
| | MNEALHNMYTQKSLSLSPGK* |
| 3 | sequence for CMV promoter coupled to intron from chicken globin |
| | splice acceptor and rabbit globin splice donor (CAG) promoter |
| | gagttccgcgttacataacttacggtaaatggcccgcctggctgaccgcccaacgacccccgcccattg |
| | acgtcaataatgacgtatgttcccatagtaacgccaatagggactttccattgacgtcaatgggtggag |
| | tatttacggtaaactgcccacttggcagtacatcaagtgtatcatatgccaagtacgccccctattgac |
| | gtcaatgacggtaaatggcccgcctggcattatgcccagtacatgaccttatgggactttcctacttgg |
| | cagtacatctacgtattagtcatcgctattaccatggtcgaggtgagccccacgttctgcttcactctc |
| | cccatctcccccccctccccacccccaattttgtatttatttattttttaattattttgtgcagcgatg |
| | ggggcggggggggggggggggcgcgcgccaggcggggcggggcggggcgaggggcggggcggggcgagg |
| | cggagaggtgcggcggcagccaatcagagcggcgcgctccgaaagtttccttttatggcgaggcggcgg |
| | cggcggcggccctataaaaagcgaagcgcgcggcgggcgggagtcgctgcgacgctgccttcgccccgt |
| | gccccgctccgccgccgcctcgcgccgcccgccccggctctgactgaccgcgttactcccacaggtgag |
| | cgggcgggacggcccttctcctccgggctgtaattagcgcttggtttaatgacggcttgtttcttttct |
| | gtggctgcgtgaaagccttgaggggctccgggagggccctttgtgcgggggggagcggctcggggggtg |
| | cgtgcgtgtgtgtgtgcgtggggagcgccgcgtgcggcccgcgctgcccggcggctgtgagcgctgcgg |
| | gcgcggcgcggggctttgtgcgctccgcagtgtgcgcgaggggagcgcggccgggggcggtgccccgcg |
| | gtgcggggggggctgcgaggggaacaaaggctgcgtgcggggtgtgtgcgtgggggggtgagcaggggg |
| | tgtgggcgcggcggtcgggctgtaacccccccctgcacccccctccccgagttgctgagcacggcccgg |
| | cttcgggtgcggggctccgtacggggcgtggcgcggggctcgccgtgccgggcggggggtggcggcagg |
| | tgggggtgccgggcggggcggggccgcctcgggccggggagggctcgggggaggggcgcggcggccccc |
| | ggagcgccggcggctgtcgaggcgcggcgagccgcagccattgccttttatggtaatcgtgcgagaggg |
| | cgcagggacttcctttgtcccaaatctgtgcggagccgaaatctgggaggcgccgccgcaccccctcta |
| | gcgggcgcggggcgaagcggtgcggcgccggcaggaaggaaatgggcggggagggccttcgtgcgtcgc |
| | cgcgccgccgtccccttctccctctccagcctcggggctgtccgcggggggacggctgccttcgggggg |
| | gacggggcagggcggggttcggcttctggcgtgtgaccggcggctctagagcctctgctaaccatgttc |
| | atgccttcttctttttcctacagctcctgggcaacgtgctggttattgtgctgtctcatcattttggca |
| | aagaattcc |
| 4 | exemplary WPRE sequence |
| | AATCAACCTCTGGATTACAAAATTTGTGAAAGATTGACTGGTATTCTTAACTATGTTGCTCCTTTTACG |
| | CTATGTGGATACGCTGCTTTAATGCCTTTGTATCATGCTATTGCTTCCCGTATGGCTTTCATTTTCTCC |
| | TCCTTGTATAAATCCTGGTTGCTGTCTCTTTATGAGGAGTTGTGGCCCGTTGTCAGGCAACGTGGCGTG |
| | GTGTGCACTGTGTTTGCTGACGCAACCCCCACTGGTTGGGGCATTGCCACCACCTGTCAGCTCCTTTCC |
| | GGGACTTTCGCTTTCCCCCTCCCTATTGCCACGGCGGAACTCATCGCCGCCTGCCTTGCCCGCTGCTGG |
| | ACAGGGGCTCGGCTGTTGGGCACTGACAATTCCGTGGTGTTGTCGGGGAAGCTGACGTCCTTTCCATGG |
| | CTGCTCGCCTGTGTTGCCACCTGGATTCTGCGCGGGACGTCCTTCTGCTACGTCCCTTCGGCCCTCAAT |
| | CCAGCGGACCTTCCTTCCCGCGGCCTGCTGCCGGCTCTGCGGCCTCTTCCGCGTCTTCGCCTTCGCCCT |
| | CAGACGAGTCGGATCTCCCTTTGGGCCGCCTCCCCGCCTGG |
| 5 | bGHpolyA (Bovine Growth Hormone Polyadenylation Signal Sequence) |
| | GTGAGATCCGCTCGCTGATCAGCCTCGACTGTGCCTTCTAGTTGCCAGCCATCTGTTGTTTGCCCCTCC |
| | CCCGTGCCTTCCTTGACCCTGGAAGGTGCCACTCCCACTGTCCTTTCCTAATAAAATGAGGAAATTGCA |
| | TCGCATTGTCTGAGTAGGTGTCATTCTATTCTGGGGGGTGGGGTGGGGCAGGACAGCAAGGGGGAGGAT |
| | TGGGAAGACAATAGCAG |

What is claimed is:

1. A recombinant viral vector comprising:
a protein coding nucleic acid that comprises a nucleotide sequence that encodes a polypeptide that comprises a soluble, ligand binding extracellular domain fragment of a Vascular Endothelial Growth Factor Receptor 3 (VEGFR3); and
a promoter operably linked to the protein coding nucleic acid, wherein said promoter is capable of promoting expression of the protein coding nucleic acid in mammalian cells, said promoter selected from the group consisting of: (a) a cytomegalovirus enhancer/chicken beta-actin/Rabbit β-globin promoter (CAG) promoter; and (b) an EF-1 alpha promoter.

2. The vector of claim 1, wherein the protein coding nucleic acid sequence comprises a polynucleotide sequence at least 90% identical to nucleotides 1-987 of SEQ ID NO: 1.

3. The vector of claim 1, wherein the protein coding nucleic acid sequence comprises a polynucleotide sequence corresponding to nucleotides 1-987 of SEQ ID NO: 1.

4. The vector of claim 1, wherein the fragment comprises the first three (1-3) Ig-like domains of human VEGFR-3.

5. The vector of claim 4, wherein the fragment comprises an amino acid sequence at least 90% identical to residues 1-328 of SEQ ID NO: 2.

6. The vector of claim 4, wherein the fragment comprises an amino acid sequence corresponding to residues 1-328 of SEQ ID NO: 2.

7. The vector of claim 4, wherein the polypeptide further includes a signal peptide.

8. The vector of claim 7, wherein the polypeptide further comprises a human IgG Fc amino acid sequence attached to the fragment of VEGFR-3.

9. The vector of claim 8, wherein the human IgG Fc amino acid sequence comprises an amino acid sequence at least 90% identical residues 342-572 of SEQ ID NO: 2.

10. The vector of claim 8, wherein the human IgG Fc amino acid sequence comprises residues 342-572 of SEQ ID NO: 2.

11. The vector of claim 8, wherein the polypeptide comprises an amino acid sequence at least 90% identical to SEQ ID NO: 2.

12. The vector of claim 8, wherein the polypeptide comprises the amino acid sequence of SEQ ID NO: 2.

13. The vector of claim 1, wherein said promoter is a cytomegalovirus enhancer/chicken beta-actin/Rabbit β-globin promoter (CAG) promoter.

14. The vector of claim 8, further comprising at least one nucleotide sequence selected from the group consisting of:
(a) functional viral 5′ and 3′ inverted terminal repeat (ITR) sequences;
(b) a Woodchuck Hepatitis Virus Post-transcriptional regulatory element (WPRE); and
(c) a bovine growth hormone polyadenylation sequence (bGHpA).

15. The vector of claim 8, that is a recombinant adeno-associated virus (rAAV) vector.

16. The vector of claim 14, wherein said vector comprises in the 5′ to 3′ direction, the 5′ ITR, the promoter, the protein coding nucleic acid, the WPRE, the poly A sequence; and the 3′ ITR.

17. The vector of claim 16, wherein the vector comprises an amino acid sequence at least 90% identical to SEQ ID NO: 6.

18. The vector of claim 16, wherein the vector comprises the amino acid sequence of SEQ ID NO: 6.

19. A method of inhibiting one or more of lymphangiogenesis, lymphatic metastasis and angiogenesis in a mammalian subject, comprising:
administering a replication-deficient vector according to claim 1 to a mammalian subject in need of treatment to inhibit one or more of lymphangiogenesis, lymphatic metastasis, and angiogenesis.

20. A method of inhibiting one or more of lymphangiogenesis, lymphatic metastasis and angiogenesis in a mammalian subject, comprising:
administering a replication-deficient vector according to claim 16 to a mammalian subject in need of treatment to inhibit one or more of lymphangiogenesis, lymphatic metastasis, and angiogenesis.

21. The method of claim 20, wherein vector is administered by at least one route selected from the group consisting of intravenous, intramuscular, and into the portal vasculature of said mammal.

22. The method of claim 20, wherein the mammal has cancer.

23. A method of treating cancer in a mammal, comprising:
administering a replication-deficient vector according to claim 1 to a mammal in need of treatment for cancer, in an amount effective to cause said vector to infect mammalian cells and cause production in the mammal of the soluble VEGFR3 polypeptide in a therapeutically effective amount.

24. A method of treating cancer in a mammal, comprising:
administering a replication-deficient vector according to claim 16 to a mammal in need of treatment for cancer, in an amount effective to cause said vector to infect mammalian cells and cause production in the mammal of the soluble VEGFR3 polypeptide in a therapeutically effective amount.

25. The method of claim 24, wherein vector is administered by at least one route selected from the group consisting of intravenous, intramuscular, and into the portal vasculature of said mammal.

26. The method according to claim 24, wherein the mammal has at least one cancer selected from the group consisting of prostate, kidney, melanoma and lung cancers.

27. A method of expressing sVEGFR3 in a mammalian cell, comprising:
administering to a mammalian cell a vector according to claim 1, to cause the cell to express the polypeptide.


SEQUENCE LISTING (6 sequences)

SEQ ID NO: 1 - DNA, 1719 bp, Homo sapiens
atgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 572 aa, Homo sapiens [sequence omitted]

SEQ ID NO: 3 - DNA, 1665 bp, Artificial sequence
  CAG promoter
gagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcc

SEQ ID NO: 4 - DNA, 593 bp, Artificial sequence
  Exemplary WPRE sequence
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgg

SEQ ID NO: 5 - DNA, 224 bp, Artificial sequence
  bGHpolyA (Bovine Growth Hormone Polyadenylation Signal Sequence)
gtgagatccgctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcag

SEQ ID NO: 6 - DNA, 8322 bp, Artificial sequence
  AAV vector
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggggctgcagcccgggggatccactagtactcgagctcaagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggagcttttcgccaccatgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagctagagcctgcggccgcaattgctagcgaatgcacttaaggatccgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtaccttgtggtgagatccgctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagtcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg